(19) United States
(12) Patent Application Publication
(10) Pub. No.: US 2022/0257805 A1
(43) Pub. Date: Aug. 18, 2022

(54) PHARMACEUTICAL FORMULATIONS AND METHODS FOR DELIVERING A THERAPEUTIC, DIAGNOSTIC, OR IMAGING AGENT TO CD206

(71) Applicants: Syracuse University, Syracuse, NY (US); Xeragenx LLC, St. Louis, MO (US)

(72) Inventors: Jonathan Bortz, St. Louis, MO (US); Robert Doyle, Syracuse, NY (US); Jayme Workinger, Syracuse, NY (US)

(21) Appl. No.: 17/613,407

(22) PCT Filed: May 20, 2020
(86) PCT No.: PCT/US2020/033749

§ 371 Date: Nov. 22, 2021

Related U.S. Application Data
(60) Provisional application No. 62/850,364, filed on May 20, 2019.
(60) Provisional application No. 62/927,528, filed on Oct. 29, 2019.

(51) Int. Cl. A61K 51/04; A61K 51/08; A61K 47/54; A61K 47/64

(52) U.S. Cl. CPC A61K 51/0451; A61K 51/0497; A61B 6/037; A61K 47/546; A61K 47/64; A61K 51/08

(57) ABSTRACT

The present disclosure provides pharmaceutical formulations and methods for delivering a therapeutic, diagnostic, or imaging agent to CD206. In an aspect, the present disclosure encompasses a pharmaceutical formulation for administration. The pharmaceutical formulation comprises a recombinantly produced intrinsic factor (IF), wherein the IF has a glycosylation pattern that enables binding to CD206.

### CROSS REFERENCE TO RELATED APPLICATIONS

This application claims priority to U.S. Provisional Application No. 62/850,364, filed May 20, 2019, and U.S. Provisional Application No. 62/927,528, filed Oct. 29, 2019, the disclosures of which are incorporated herein by reference.

### FIELD OF THE INVENTION

The present disclosure provides pharmaceutical formulations and methods for delivering a therapeutic, diagnostic, or imaging agent to CD206.

### BACKGROUND OF THE INVENTION

A basic understanding of the dietary pathway of vitamin B12 (B12) is in place. Mammals have a complex dietary uptake pathway for B12 involving a series of transport proteins and specific receptors across various tissues and organs. Transport and delivery of B12 utilizes three primary carrier proteins: haptocorrin (HC; Kd=0.01 pM), intrinsic factor (IF; Kd=1 pM), and transcobalamin (TC; Kd=0.005 pM), each responsible for carrying a single B12 molecule. B12 is initially released from food by the action of peptic enzymes and the acidic environment of the gastrointestinal system and then bound by HC (Holo-HC). Holo-HC travels from the stomach to the duodenum, where pancreatic digestion effects B12 release, whereupon it is bound by gastric intrinsic factor (IF). IF is a ˜50 kDa glycosylated protein that is secreted from parietal cells of the gastric mucosa and is resistant to pancreatic enzymes. Once B12 is bound to IF, it typically facilitates intestinal transport and passage across the ileal enterocyte. This passage occurs via receptor-mediated endocytosis through the IF-B12 receptor cubilin (CUBN) combined with a transmembrane protein amnionless. Following internalization, IF is degraded by lysosomal proteases and B12 is released into the blood stream, either as free B12 or pre-bound to TC. Cells that require B12 express the holo-TC receptor, CD320. Upon internalization, TC is degraded and B12 is transported from the lysosome for cellular use.

The present disclosure details the discovery that recombinantly produced glycosylated IF targets a different in vivo pathway, and therefore, may be used as a delivery mechanism.

### SUMMARY OF THE INVENTION

In an aspect, the present disclosure encompasses a pharmaceutical formulation for administration. The pharmaceutical formulation comprises a recombinantly produced intrinsic factor (IF), wherein the IF has a glycosylation pattern that enables binding to CD206. In some embodiments, the IF is conjugated to a therapeutic, diagnostic, or imaging agent. In some embodiments, the pharmaceutical formulation comprises B12 or B12 analog, optionally wherein the B12 or B12 analog is conjugated to a therapeutic, diagnostic, or imaging agent. In further embodiments, the IF is complexed to the B12, B12 analog, B12 conjugate, or B12 analog conjugate.

In another aspect, the present disclosure encompasses a pharmaceutical formulation for systemic administration. The pharmaceutical formulation comprises a recombinantly produced intrinsic factor (IF), wherein the IF has a glycosylation pattern that enables binding to CD206, and is conjugated to a therapeutic, diagnostic, or imaging agent.

In another aspect, the present disclosure encompasses a method of delivering a therapeutic, diagnostic, or imaging agent to the liver of a subject. The method comprises administering to the subject a pharmaceutical formulation comprising a recombinantly produced intrinsic factor (IF), wherein the IF is conjugated to a therapeutic, diagnostic, or imaging agent and binds to CD206 in the liver of the subject.

In another aspect, the present disclosure encompasses a method of delivering a therapeutic, diagnostic, or imaging agent to the liver of a subject. The method comprises administering to the subject a pharmaceutical formulation comprising (a) a recombinantly produced intrinsic factor (IF), wherein the IF binds to CD206 in the liver of the subject, and (b) B12 or B12 analog conjugated to a therapeutic, diagnostic, or imaging agent. In some embodiments, the pharmaceutical formulation comprises IF complexed to the B12 conjugate or the B12 analog conjugate.

In yet another aspect, the present disclosure encompasses a method of treating microbial infection, inflammation or cancer in a subject. The method comprises administering to the subject a pharmaceutical formulation comprising a recombinantly produced intrinsic factor (IF), wherein the IF is conjugated to a therapeutic, diagnostic, or imaging agent and binds to CD206 in the liver, on macrophages, on immature dendritic cells, or on skin epithelia of the subject. In some embodiments, the IF binds to liver cells. In some embodiments, the IF binds to macrophages. In some embodiments, the pharmaceutical formulation is administered by inhalation and IF binds to alveolar macrophages.

In yet another aspect, the present disclosure encompasses a method of treating microbial infection, inflammation or cancer in a subject. The method comprises administering to the subject a pharmaceutical formulation comprising (a) a recombinantly produced intrinsic factor (IF), wherein the IF binds to CD206 in the liver, on macrophages, on immature dendritic cells, or on skin epithelia of the subject, and (b) B12 or B12 analog conjugated to a therapeutic, diagnostic, or imaging agent. In some embodiments, the pharmaceutical formulation comprises IF complexed to the B12 conjugate or the B12 analog conjugate. In certain embodiments, the IF binds to liver cells expressing CD206. In certain embodiments, the IF binds to macrophages expressing CD206. In further embodiments, the pharmaceutical formulation is administered by inhalation and the IF binds to alveolar macrophages expressing CD206.

In still another aspect, the present disclosure encompasses a method of delivering a therapeutic, diagnostic, or imaging agent to a cell that expresses CD206 in a subject. The method comprises administering a pharmaceutical formulation to the subject comprising a recombinantly produced intrinsic factor (IF), wherein the IF has a glycosylation pattern that enables binding to CD206, and is conjugated to a therapeutic, diagnostic, or imaging agent. In some embodiments, the cell is a liver cell expressing CD206. In some embodiments, the cell is a macrophage expressing CD206. In some embodiments, the pharmaceutical formulation is administered by inhalation and the cell is an alveolar macrophage expressing CD206.

In still another aspect, the present disclosure encompasses a method of delivering a therapeutic, diagnostic, or imaging agent to a cell that expresses CD206 in a subject. The method comprises administering a pharmaceutical formulation to the subject comprising (a) a recombinantly produced intrinsic factor (IF), wherein the IF has a glycosylation pattern that enables binding to CD206, and (b) B12 or B12 analog conjugated to a therapeutic, diagnostic, or imaging agent. In some embodiments, the pharmaceutical formulation comprises IF complexed to the B12 conjugate or the B12 analog conjugate. In certain embodiments, the cell is a liver cell expressing CD206. In certain embodiments, the cell is a macrophage expressing CD206. In further embodiments, the pharmaceutical formulation is administered by inhalation and the cell is an alveolar macrophage expressing CD206.

In a further aspect, the present disclosure encompasses a method of modulating CD206 function. The method comprises administering a pharmaceutical formulation comprising a recombinantly produced intrinsic factor (IF), wherein the IF has a glycosylation pattern that enables binding to CD206, and is conjugated to a therapeutic, diagnostic, or imaging agent to a subject.

In a further aspect, the present disclosure encompasses a method of modulating CD206 function. The method comprises administering a pharmaceutical formulation to the subject comprising (a) a recombinantly produced intrinsic factor (IF), wherein the IF has a glycosylation pattern that enables binding to CD206, and (b) B12 or B12 analog conjugated to a therapeutic, diagnostic, or imaging agent. In some embodiments, the pharmaceutical formulation comprises IF complexed to the B12 conjugate or the B12 analog conjugate.

In an additional aspect, the present disclosure encompasses a method of detecting microbial infection, inflammation or cancer in a subject. The method comprises administering to the subject a pharmaceutical formulation comprising a recombinantly produced intrinsic factor (IF), wherein the IF has a glycosylation pattern that enables binding to CD206, and is conjugated to an imaging agent; and detecting the imaging agent, wherein the presence of the imaging agent indicates the presence of microbial infection, arthritis or cancer in the subject.

In an additional aspect, the present disclosure encompasses a method of detecting microbial infection, inflammation or cancer in a subject. The method comprises administering to the subject a pharmaceutical formulation comprising (a) a recombinantly produced intrinsic factor (IF), wherein the IF has a glycosylation pattern that enables binding to CD206, and (b) B12 or B12 analog conjugated to an imaging agent; and detecting the imaging agent, wherein the presence of the imaging agent indicates the presence of microbial infection, arthritis or cancer in the subject. In some embodiments, the pharmaceutical formulation comprises IF complexed to the B12 conjugate or the B12 analog conjugate.

In another aspect, the present disclosure encompasses a method of treating microbial infection, arthritis or cancer in a subject. The method comprising administering to the subject a pharmaceutical formulation comprising a recombinantly produced intrinsic factor (IF), wherein the IF has a glycosylation pattern that enables binding to CD206, and is conjugated to a therapeutic agent.

In another aspect, the present disclosure encompasses a method of treating microbial infection, arthritis or cancer in a subject. The method comprising administering to the subject a pharmaceutical formulation comprising (a) a recombinantly produced intrinsic factor (IF), wherein the IF has a glycosylation pattern that enables binding to CD206, and (b) B12 or B12 analog conjugated to an imaging agent; and detecting the imaging agent, wherein the presence of the imaging agent indicates the presence of microbial infection, arthritis or cancer in the subject. In some embodiments, the pharmaceutical formulation comprises IF complexed to the B12 conjugate or the B12 analog conjugate.

In yet another aspect, the present disclosure encompasses a method of delivering B12 to a cell that expresses CD206 in a subject, the method comprising administering a pharmaceutical formulation to the subject comprising a recombinantly produced intrinsic factor (IF), wherein the IF has a glycosylation pattern that enables binding to CD206, and is conjugated to B12.

In yet another aspect, the present disclosure encompasses a method of delivering B12 to a cell that expresses CD206 in a subject, the method comprising administering a pharmaceutical formulation to the subject comprising a recombinantly produced intrinsic factor (IF), wherein the IF has a glycosylation pattern that enables binding to CD206, and is complexed to B12 or a B12 analog.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts the dietary uptake pathway for B12 (Cbl) in humans and proposed experimental design. Gastric intrinsic factor (IF) was injected systemically (Boxed; Star-Cbl-IF) to investigate biodistribution. Note, B12 may also be released into blood as free B12.

FIG. 2 depicts binding affinities of ⁹¹Zr-B12 and CN-B12 to human gastric IF with a K_{d} observed of 1.57 nM and 1.36 nM, respectively.

FIG. 3A and FIG. 3B depict flow cytometry analysis in (FIG. 3A) BN16 cells treated with IF-B12-Cy5 (orange), B12-Cy5 (blue) (200 nM each) at 37° C. and IF-B12-Cy5 (200 nM) at 4° C. (green) in HBSS for 1 h. Untreated cell background fluorescence is indicated in red; (FIG. 3B) J774A.1 cells treated with IF-B12-Cy5 (orange), B12-Cy5 (blue) (200 nM each) and IF-B12-Cy5 cells with mannan block (2 mg/mL) in HBSS for 1 h at 37° C.

FIG. 4A and FIG. 4B depict PET images of representative nude athymic mice on B12 replete (FIG. 4A) and deplete (FIG. 4B) diets after injections of ⁸⁹Zr-B12 or IF-⁸⁹Zr-B12 at 5 and 24 h p.i.

FIG. 5A and FIG. 5B depict ex vivo tissue distribution of ⁸⁹Zr-B12 (FIG. 5A) and IF-⁸⁹Zr-B12 (FIG. 5B) in mice (n 3) on a B12 deplete or replete diet at 24 h plotted as % recovered/organ mean±SD. ⁸⁹Zr-B12 showed significant changes occurred in liver, kidneys, blood, pancreas, and heart between the two mice models (liver: 32.18±2.6 vs. 36.24±1.8, kidney: 53.58±2.7 vs. 48.89±1.0, blood: 1.60±1.0 vs. 0.192±0.05, pancreas: 0.489±0.18 vs. 1.19±0.15, heart: 0.740±0.14 vs. 0.501±0.05, % recovered/organ for replete vs. deplete; p≤0.05). IF-⁸⁹Zr-B12 showed significant changes occurred in blood, and heart (blood: 0.69±0.31 vs. 0.106±0.01, heart: 0.51±0.09 vs. 0.23±0.04% recovered/organ for replete vs. deplete; p 0.05).

FIG. 6 depicts ex vivo tissue distribution of IF-⁸⁹Zr-B12 and ⁸⁹Zr-B12 in mice on a B12 deplete or replete diet at 24 h plotted as % recovered/organ as mean±SD. The significant changes occurred with ⁸⁹Zr-B12 in the liver and kidney, while they were not significantly changed in the IF-⁸⁹Zr-B12. n≥3, *p≤0.05.

FIG. 7 depicts MALDI-MS analysis of B12-DFO bound to cold Zr⁴⁺. Expected: 2030.2 [M⁺]; observed: 2005.2 [M-CN+H]⁺.

FIG. 8 depicts iTLC of IF-⁸⁹Zr-B12 solution after 30 min incubation with a 1:0.8 excess of IF to ⁸⁹Zr-B12. Results indicate all ⁸⁹Zr-B12 was bound by IF and no loss of ⁸⁹Zr was observed.

FIG. 9 depicts iTLC of IF-⁸⁹Zr-B12 stability at 0, 1, 4, and 24 h incubation with saline at 37° C. Results indicated complex was stable with no loss of tracer noted. Shift in peak was attributed to unaligned spotting.

FIG. 10 depicts Western results for cubilin in CHO and BN16 cells showing cubilin expression in BN16 cells and no expression in CHO cells. Lane 1: Thermo Fisher Scientific HiMark Pre-Stained HMW Protein Standard, lane 2: CHO-K1 cell lysate, lane 3: BN16 cell lysate. 1° Ab: Santa Cruz Biotechnology cubilin anti-goat polyclonal (1:200), 2° Ab: Santa Cruz Biotechnology chicken anti-goat HRP conjugated (1:4000).

FIG. 11 depicts Western blot results for ASGPR in HepG2 cells. Expression was seen in HepG2 cells and not CHO cells. Lane 1: CHO-K1 lysate, Lane 2: BioRad Kaleidoscope Protein Markers, Lane 3: HEPG2 Cell lysate was ran on a 12% agarose gel and transferred on a PDVF membrane. Blocked for 1 h and the primary antibody-HRP: 1:200 overnight at 4° C.

FIG. 12 depicts flow cytometry results for uptake of IF-B12-Cy5 and B12-Cy5 in CHO-K1 cells. Neither compound was internalized by CHO-K1 cells indicating no expression of cubilin or CD206. Analysis on a Becton Dickinson LSRII Cell Analyzer. Excitation: 640 nm, Emission: 660/20. Solutions were prepared at 100 nM in HBSS. Red: untreated, Blue: B12-Cy5, Orange: IF-B12-Cy5.

FIG. 13 depicts flow cytometry results for uptake of IF-B12-Cy5 and B12-Cy5 in HepG2 cells. Both compounds were not internalized by HepG2 cells indicating no recognition by any cell receptors. Analysis on a Becton Dickinson LSRII Cell Analyzer. Excitation: 640 nm, Emission: 660/20. Solutions were prepared at 100 nM in HBSS. Red: untreated, Blue: B12-Cy5, Orange: IF-B12-Cy5.

FIG. 14A and FIG. 14B depict liver IHC analysis of (FIG. 14A) PBS control and (FIG. 14B) CD206 imaged liver slices at 40×. Black arrows outlined in white indicate areas where there is cell surface specific binding. Arrows in blue outlined in white indicate nuclear localization of the antibody. Tissues were sectioned at 5 pM and stained with an anti-mannose receptor antibody at a 1:100 dilution from stock.

FIG. 15 depicts the synthesis of IF-⁸⁹Zr-B12. The B12-DFO conjugate was first incubated with ⁸⁹Zr at neutral pH at room temperature for 15 min. After confirmation of binding through iTLC, ⁸⁹Zr-B12-DFO was incubated with a slight excess of apo-IF (indicated in red) for 30 min then purified with a 30 kDa spin filter (GE Vivaspin).

FIG. 16A and FIG. 16B depict the synthesis of a B12 conjugate comprising a chloroquine derivative.

FIG. 17 depicts the results from an NMR analysis confirming the synthesis of a B12 conjugate comprising a chloroquine derivative.

### DETAILED DESCRIPTION OF THE INVENTION

Among the various aspects of the disclosure is the provision of a pharmaceutical formulation comprising IF from Arabidopsis conjugated to a therapeutic, diagnostic, or imaging agent. The present disclosure also provides a pharmaceutical formulation comprising B12, or an analog of B12, conjugated to a therapeutic, diagnostic, or imaging agent, and IF from Arabidopsis. Other aspects of the pharmaceutical formulations are detailed below. The pharmaceutical formulations of the disclosure may be used to target CD206 expressing cells. Such pharmaceutical formulations may also be used to deliver a therapeutic, diagnostic, or imaging agent to CD206 expressing cells and image and/or treat microbial infections, inflammation, arthritis or cancer. Prior to this disclosure, the chemical composition of the glycosylation pattern of IF produced from Arabidopsis was unknown. Further, it was unknown that this glycosylation pattern would facilitate binding of IF to CD206.

### I. Composition

The present invention encompasses a pharmaceutical formulation comprising intrinsic factor (IF), optionally B12 or B12 analog, and at least one therapeutic, diagnostic, or imaging agent, wherein a therapeutic, diagnostic, or imaging agent is conjugated to the IF, B12, or B12 analog. In some embodiments, a pharmaceutical formulation may comprise a B12 analog. Such analogs may be modified to improve bioavailability, solubility, stability, handling properties, or a combination thereof, as compared to an unmodified version. Thus, in another aspect, a pharmaceutical formulation of the disclosure may comprise a modified B12 or B12 analog. In still another aspect, a pharmaceutical formulation of the disclosure may comprise a prodrug of B12 or a B12 analog.

A pharmaceutical formulation of the disclosure may further comprise a pharmaceutically acceptable excipient, carrier or diluent. Further, a pharmaceutical formulation of the disclosure may contain preserving agents, solubilizing agents, stabilizing agents, salts (substances of the present invention may themselves be provided in the form of a pharmaceutically acceptable salt), buffers, or antioxidants.

### (a) Vitamin B12 (Cobalamin)

Vitamin B12 is a water-soluble vitamin with a highly complex structure, comprising a midplanar corrin ring composed of four pyrroline elements linked to a central cobalt(III) atom. Throughout the disclosure vitamin B12, B12 and cobalamin may be used interchangeably.

In the structure of vitamin B12, the central cobalt(III) atom is six-coordinated, with the equatorial positions filled by the nitrogen atoms of the corrin macrocycle. The (conventionally) ‘lower’, ‘α’-axial site is occupied by an imidazole nitrogen atom from a 5′,6′-dimethylbenzimidazole (DMB) base whereas the ‘upper’, ‘β’-axial site can be occupied by various X groups (e.g. CN⁻, CH₃⁻, Ado⁻, SCN⁻, SeCN⁻, SO₃⁻ and thiourea). The corrin ring incorporates seven amide side chains, three acetamides (a, c, g) and four propionamides (b, d, e, f). The four pyrrole rings are usually indicated as A, B, D and D.

Several functional groups are readily available for modification on B12, including propionamides, acetamides, hydroxyl groups, the cobalt(III) ion and the phosphate moiety. Accordingly, a B12 conjugate of the invention may be modified at a propionamide, acetamide, hydroxyl group, the cobalt(III) ion and the phosphate moiety, provided the B12 conjugate binds IF. Non-limiting examples of modification sites for a B12 conjugate of the disclosure include at the a-position or b-position on the A-ring, at the c-position or d-position on the B-ring, at the e-position on the C-ring, at the g-position on the D ring, at the f-position, at the phosphate moiety, at the 5′- or 2′-hydroxyl on the ribose, and at the cobalt ion. Preferred sites of modification may include sites on the A ring such as the b-position, sites on the C ring such as the e-position, sites on the ribose unit such as the 5′-hydroxyl group, and the cobalt cation. Specifically, the e-position may be modified to allow interaction with IF. Alternatively, the b-position may be modified to disrupt TC binding specifically. However, other sites of modification may be utilized provided they maintain the binding affinity of B12 for IF.

Methods for modification to B12 are known in the art. The following provides non-limiting examples of methods for modification. It is contemplated that various other methods for modification common in the art of synthetic chemistry may be used. For example, carefully controlled partial hydrolysis of cyanocobalamin under acidic conditions gives access to desirable b and e acids. Methods for 5′-OH functionalization may rely on the reaction of cyanocobalamin ((CN)Cbl) with anhydrides, furnishing unstable ethers. Another method for conjugation may be the carbamate or carbonate methodology as described by Russell-Jones (WO 1999/065390, which is hereby incorporated by reference in its entirety). Briefly, the hydroxyl group at position 5′ is first reacted with a carbonyl group equivalent—1,1′-carbonyldiimidazole (CDI) or 1,1′-carbonylbis(1,2,4-triazole) (CDT)—and then treated with an amine or an alcohol giving carbamates and carbonates, respectively, at the 5′-position of the ribose tail. Alternatively, the 5′-OH group can be oxidized to the corresponding carboxylic acid using the 2-iodoxybenzoic acid (IBX)/2-hydroxypyridine (HYP) system as an oxidant and then coupled with amines. Another effective approach may rely on [1,3] dipolar cycloaddition. The 5′-OH is transformed into a good leaving group and subsequently substituted with an azide. The resulting “clickable” azide is stable and highly active in the copper-catalyzed as well as in the strain promoted [1,3] dipolar cycloaddition (CuAAC or SPAAC) to alkynes. This methodology is described in detail in Chrominski et al, Chem Eur J 2013; 19: 5141-5148, which is hereby incorporated by reference in its entirety. In a specific embodiment, the 5′-OH is transformed into an azide. An alkyne containing glycine is then added using “click” chemistry, which may then be chelated to a metal. In another specific embodiment, an alkyne comprising glycine is added at the b-position, which may be then be chelated to a metal. In still yet another specific embodiment, an alkyl chain linker may added be prior to the group responsible for metal chelation.

Functionalization of the cobalt ion may be accomplished by either alkylation or utilization of cyanide ligand properties to act as an electron pair donor for transition metals, resulting in bimetallic complexes. The synthesis of organometallic species requires reduction of the cobalt(III) to cobalt(I) B12 and its subsequent reaction with electrophiles: alkyl halides, acyl halides, Michael acceptors, epoxides, etc. Alternatively, reduction may not be required and instead, the direct reaction of (CN)Cbl with terminal alkynes in the presence of Cu(I) salts may furnish acetylides in excellent yields. This methodology may allow the conjugation of two moieties to B12 and is described in further detail in Chrominski et al, J Org Chem 2014; 79: 7532-7542, which is hereby incorporated by reference in its entirety. Accordingly, it is contemplated that two imaging agents and/or therapeutic agents may be conjugated to B12. Briefly, using this methodology, “doubly clickable” vitamin B12, a valuable building block for further functionalization via [1,3] dipolar azide-alkyne cycloaddition, may be prepared. A combination of AAC (CuAAC and SPAAC) with the carbamate method may allow conjugation at both the central cobalt ion and the 5′-position. In an embodiment, an alkyne comprising glycine may be added at the cobalt ion, which may then be chelated to a metal. In another embodiment, an alkyl chain linker may or may not be added prior to the group responsible for metal chelation.

B12 or an analog thereof and an imaging agent and/or therapeutic agent may be: i) conjugated directly together; ii) held apart by a ‘linker’ to produce distance between the B12 or an analog thereof and the imaging agent and/or therapeutic agent; or iii) conjugated to carriers that can couple the desired imaging agent and/or therapeutic agent unconjugated, within the carrier. Suitable imaging, diagnostic, and therapeutic agents are described in more detail below and in Section I(b) and Section I(c).

In an aspect, B12 or an analog thereof may be conjugated to an imaging agent and/or therapeutic agent directly via a covalent bond or indirectly via charge interaction. Non-limiting examples of a charge interaction may include ionic, hydrophobic, hydrogen bonding or Van der Waals forces. In an embodiment where B12 or an analog thereof is coupled directly to an imaging agent and/or therapeutic agent, a linker may or may not be used.

In another aspect, B12 or an analog thereof may be conjugated to a carrier that can couple the desired imaging agent and/or therapeutic agent unconjugated, within the carrier. Non-limiting examples of suitable carriers may include chelating agents. For example, B12 or an analog thereof may be conjugated to a chelating agent that can couple the desired imaging agent and/or therapeutic agent. The chelating agent may be directly conjugated to B12 or an analog thereof or may be conjugated to a linker that is conjugated to B12 or an analog thereof. As used herein, a “chelating agent” is a molecule that forms multiple chemical bonds with a single metal atom. Prior to forming the bonds, the chelating agent has more than one pair of unshared electrons. The bonds are formed by sharing pairs of electrons with the metal atom.

Examples of chelating agents include, but are not limited to, iminodicarboxylic and polyaminopolycarboxylic reactive groups, diethylenetriaminepentaacetic acid (DTPA), 1,4,7,10-tetraazacyclododecane-1,4,7,10-tetraacetic acid (DOTA), tetramethyl heptanedionate (TMHD), 2,4-pentanedione, ethylenediamine-tetraacetic acid disodium salt (EDTA), ethyleneglycol-0,0′-bis(2-aminoethyl)-N,N,N′,N′-tetraacetic acid (EGTA), N-(2-hydroxyethyl)ethylenediamine-N,N′,N′-triacetic acid trisodium salt (HEDTA), nitrilotriacetic acid (NTA), and 1,4,8,11-tetraazacyclotetradecane-N,N′,N″,N′″-tetraacetic acid (TETA), deferoxamine (DFO), 1,4,7-triazacyclononane-1,4,7-triacetic acid (NOTA), organic acids and amino acids such as citric acid, tartaric acid, gluconic acid and glycine, and derivatives thereof. In a specific embodiment, the chelating agent is DFO.

Chelating agents may be attached to B12 or an analog thereof using methods generally known in the art. The following provides non-limiting examples of methods to attach chelating agents. It is contemplated that various other methods for attaching chelating agents common in the art of synthetic chemistry may be used. For example, B12 or an analog thereof may be conjugated to a chelating agent by reacting a free amino group of B12 or an analog thereof with an appropriate functional group of the chelator, such as a carboxyl group or activated ester. For example, B12 or an analog thereof may be coupled to the chelator ethylenediaminetetraacetic acid (EDTA), common in the art of coordination chemistry, when functionalized with a carboxyl substituent on the ethylene chain. Synthesis of EDTA derivatives of this type are reported in Arya et al., (Bioconjugate Chemistry. 2:323, 1991), wherein the four coordinating carboxyl groups are each blocked with a t-butyl group while the carboxyl substituent on the ethylene chain is free to react with the amino group of B12 or an analog thereof thereby forming a conjugate.

B12 or an analog thereof may be coupled to a metal chelator component that is peptidic, i.e., compatible with solid-phase peptide synthesis. In this case, the chelator may be coupled to B12 or an analog thereof in the same manner as EDTA described above.

B12 or an analog thereof may be complexed, through its attached chelating agent, to an imaging agent, thereby resulting in a B12 or an analog thereof conjugate that is indirectly labeled. Similarly, cytotoxic or therapeutic agents may also be attached via a chelating group to B12 or an analog thereof. As such, the chelating agent may be conjugated directly to the imaging agent or therapeutic agent. Alternatively, an intervening amino acid sequence or linker can be used to conjugate the imaging agent or therapeutic agent to the chelating agent.

In another aspect, B12 or an analog thereof and the imaging agent and/or therapeutic agent may be held apart by a linker to produce distance between the B12 or an analog thereof and the imaging agent and/or therapeutic agent. It is to be understood that conjugation of the B12 or an analog thereof to the imaging agent and/or therapeutic agent will not adversely affect either the binding function of the B12 or an analog thereof to IF or the function of the imaging agent and/or therapeutic agent. Suitable linkers include, but are not limited to, amino acid chains and alkyl chains functionalized with reactive groups for conjugating to both the B12 or analog thereof and the imaging agent and/or therapeutic agent.

In an embodiment, the linker may include amino acid side chains, referred to as a peptide linker. Accordingly, amino acid residues may be added to B12 or an analog thereof for the purpose of providing a linker by which B12 or an analog thereof can be conveniently affixed to an imaging agent and/or therapeutic agent, or carrier. Amino acid residue linkers are usually at least one residue and can be 40 or more residues, more often 1 to 10 residues. Typical amino acid residues used for linking are tyrosine, cysteine, lysine, glutamic and aspartic acid, or the like.

In another embodiment, an alkyl chain linking group may be conjugated to B12 or an analog thereof. For example, by reacting an amino group of B12 or an analog thereof with a first functional group on the alkyl chain, such as a carboxyl group or an activated ester. Subsequently a chelator may be attached to the alkyl chain to complete the formation of a complex by reacting a second functional group on the alkyl chain with an appropriate group on the chelator. The second functional group on the alkyl chain is selected from substituents that are reactive with a functional group on the chelator while not being reactive with B12 or an analog thereof. For example, when the chelator incorporates a functional group, such as a carboxyl group or an activated ester, the second functional group of the alkyl chain linking group can be an amino group. It will be appreciated that formation of the conjugate may require protection and deprotection of the functional groups present in order to avoid formation of undesired products. Protection and deprotection are accomplished using protecting groups, reagents, and protocols common in the art of organic synthesis. It will be appreciated that linking groups may alternatively be coupled first to the chelator and then to B12 or an analog thereof. An alkyl chain linking group may be one to 40 or more carbons long, more often 1 to 10 carbons long. In a specific embodiment, an alkyl chain linking group may be 1, 2, 3, 4, 5, 6 or 7 carbons long. In another specific embodiment, an alkyl chain linking group may be 3 carbons long. In still another specific embodiment, an alkyl chain linking group may be 4 carbons long. In yet still another specific embodiment, an alkyl chain linking group may be 5 carbons long.

An alternative chemical linking group to an alkyl chain is polyethylene glycol (PEG), which is functionalized in the same manner as the alkyl chain described above for incorporation in the conjugates. B12 or an analog thereof may be PEGylated for improved systemic half-life and reduced dosage frequency. In an embodiment, PEG may be added to a linker. As such, B12 or an analog thereof may comprise a linker and PEG. For example, B12 or an analog thereof may comprise an alkyl linker, one or more chelators and PEG.

### (b) Imaging Agent

In an aspect, a pharmaceutical composition of the present disclosure may comprise an imaging agent. Such an imaging agent may be conjugated to IF or to B12 or an analog thereof. The imaging agent may be directly conjugated to IF, B12, or an analog thereof or may be indirectly conjugated to IF, B12, or an analog thereof. In an embodiment, the imaging agent may be complexed with a chelating agent that is conjugated to IF, B12, or an analog thereof. In another embodiment, the imaging agent may be complexed with a chelating agent that is conjugated to a linker that is conjugated to IF, B12, or an analog thereof. In still another embodiment, the imaging agent may be conjugated to a linker that is conjugated to IF, B12, or an analog thereof. In still yet another embodiment, an imaging agent may be indirectly attached to IF, B12, or an analog thereof by the ability of the label to be specifically bound by a second molecule. One example of this type of an indirectly attached label is a biotin label that can be specifically bound by the second molecule, streptavidin. Single, dual or multiple labeling may be advantageous.

As used herein, an “imaging agent” is any type of agent which, when attached to IF, B12, or an analog thereof renders IF, B12, or the analog thereof detectable. An imaging agent may also be toxic to cells or cytotoxic. Accordingly, an imaging agent may also be a therapeutic agent or cytotoxic agent. In general, imaging agents may include luminescent molecules, chemiluminescent molecules, fluorochromes, fluorophores, fluorescent quenching agents, colored molecules, radioisotopes, radionuclides, cintillants, massive labels such as a metal atom (for detection via mass changes), biotin, avidin, streptavidin, protein A, protein G, antibodies or fragments thereof, Grb2, polyhistidine, Ni²⁺, Flag tags, myc tags, heavy metals, enzymes, alkaline phosphatase, peroxidase, luciferase, electron donors/acceptors, acridinium esters, and colorimetric substrates. The skilled artisan would readily recognize other useful imaging agents that are not mentioned above, which may be employed in the operation of the present invention.

An imaging agent emits a signal that can be detected by a signal transducing machine. In some cases, the imaging agent can emit a signal spontaneously, such as when the imaging agent is a radionuclide. In other cases the imaging agent emits a signal as a result of being stimulated by an external field such as when the imaging agent is a relaxivity metal. Examples of signals include, without limitation, gamma rays, X-rays, visible light, infrared energy, and radiowaves. Examples of signal transducing machines include, without limitation, gamma cameras including SPECT/CT devices, PET scanners, fluorimeters, and Magnetic Resonance Imaging (MRI) machines. As such, the imaging agent comprises a label that can be detected using magnetic resonance imaging, scintigraphic imaging, ultrasound, or fluorescence. In a specific embodiment, the imaging agent comprises a label that can be detected using positron emission tomography, single photon emission computed tomography, gamma camera imaging, or rectilinear scanning.

Suitable fluorophores include, but are not limited to, fluorescein isothiocyante (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes. B12 or an analog thereof can be labeled for fluorescence detection by labeling the agent with a fluorophore using techniques well known in the art (see, e.g., Lohse et al., Bioconj Chem 8:503-509 (1997)). For example, many known dyes are capable of being coupled to NH₂-terminal groups. Alternatively, a fluorochrome such as fluorescein may be bound to a lysine residue of a peptide linker. In a specific embodiment, an alkyne modified dye, such an Alexa Fluor dye, may be clicked to an azido modified B12 using, for example, Sharpless click chemistry (Kolb et al., Angew Chem Int Ed 2001; 40: 2004-2021, which incorporated by reference in its entirety).

A radionuclide may be a γ-emitting radionuclide, Auger-emitting radionuclide, β-emitting radionuclide, an α-emitting radionuclide, or a positron-emitting radionuclide. A radionuclide may be an imaging agent and/or a therapeutic agent. Non-limiting examples of suitable radionuclides may include carbon-11, nitrogen-13, oxygen-15, fluorine-18, fluorodeoxyglucose-18, phosphorous-32, scandium-47, copper-64, 65 and 67, gallium-67 and 68, bromine-75, 77 and 80m, rubidium-82, strontium-89, zirconium-89, yttrium-86 and 90, ruthenium-95, 97,103 and 105, rhenium-99m, 101, 105, 186 and 188, technetium-99m, rhodium-105, mercury-107, palladium-109, indium-111, silver-111, indium-113m, lanthanide-114m, tin-117m, tellurium-121m, 122m and 125m, iodine-122, 123, 124, 125, 126, 131 and 133, praseodymium-142, promethium-149, samarium-153, gadolinium-159, thulium-165, 167 and 168, dysprosium-165, holmium-166, lutetium-177, rhenium-186 and 188, iridium-192, platinum-193 and 195m, gold-199, thallium-201, titanium-201, astatine-211, bismuth-212 and 213, lead-212, radium-223, actinium-225, and nitride or oxide forms derived there from. In a specific embodiment, a radionuclide is selected from the group consisting of copper-64, zirconium-89, yttrium-86, yttrium-90, technetium-99m, iodine-125, iodine-131, lutetium-177, rhenium-186 and rhenium-188.

A variety of metal ions may be used as an imaging agent. For instance, the metal ion may be a calcium ion, scandium ion, titanium ion, vanadium ion, chromium ion, manganese ion, iron ion, cobalt ion, nickel ion, copper ion, zinc ion, gallium ion, germanium ion, arsenic ion, selenium ion, bromine ion, krypton ion, rubidium ion, strontium ion, yttrium ion, zirconium ion, niobium ion, molybdenum ion, technetium ion, ruthenium ion, rhodium ion, palladium ion, silver ion, cadmium ion, indium ion, tin ion, antimony ion, tellurium ion, iodine ion, xenon ion, cesium ion, barium ion, lanthanum ion, hafnium ion, tantalum ion, tungsten ion, rhenium ion, osmium ion, iridium ion, platinum ion, gold ion, mercury ion, thallium ion, lead ion, bismuth ion, francium ion, radium ion, actinium ion, cerium ion, praseodymium ion, neodymium ion, promethium ion, samarium ion, europium ion, gadolinium ion, terbium ion, dysprosium ion, holmium ion, erbium ion, thulium ion, ytterbium ion, lutetium ion, thorium ion, protactinium ion, uranium ion, neptunium ion, plutonium ion, americium ion, curium ion, berkelium ion, californium ion, einsteinium ion, fermium ion, mendelevium ion, nobelium ion, or lawrencium ion. In some embodiments, the metal ion may be selected from the group comprising alkali metals with an atomic number greater than twenty. In other embodiments, the metal ion may be selected from the group comprising alkaline earth metals with an atomic number greater than twenty. In one embodiment, the metal ion may be selected from the group of metals comprising the lanthanides. In another embodiment, the metal ion may be selected from the group of metals comprising the actinides. In still another embodiment, the metal ion may be selected from the group of metals comprising the transition metals. In yet another embodiment, the metal ion may be selected from the group of metals comprising the poor metals. In other embodiments, the metal ion may be selected from the group comprising gold ion, bismuth ion, tantalum ion, and gadolinium ion. In preferred embodiments, the metal ion may be selected from the group comprising metals with an atomic number of 53 (i.e. iodine) to 83 (i.e. bismuth). In an alternative embodiment, the metal ion may be an ion suitable for magnetic resonance imaging. In another alternative embodiment, the metal ion may be selected from the group consisting of metals that have a K-edge in the X-ray energy band of CT. Preferred metal ions include, but are not limited to, manganese, iron, gadolinium, gold, and iodine.

In some embodiments, a suitable metal ion may be a magnetic ion. In other embodiments, a suitable metal ion may be part of a metal nanoparticle.

The metal ion may be a metal ion in the form of +1, +2, or +3 oxidation states. For instance, non-limiting examples include Ba²⁺, Bi³⁺, Cs⁺, Ca²⁺, Cr²⁺, Cr³⁺, Cr⁶⁺, CO²⁺, Co³, Cu⁺, Cu²⁺, Cu³⁺, Ga³⁺, Gd³⁺, Au⁺, Au³⁺, Fe²⁺, Fe³⁺, F³⁺, Pb²⁺, Mn²⁺, Mn³⁺, Mn⁴⁺, Mn⁷⁺, Hg²⁺, Ni²⁺, Ni³⁺, Ag⁺, Sr²⁺, Sn²⁺, Sn⁴⁺, and Zn²⁺. The metal ion may be part of a metal oxide. For instance, non-limiting examples of metal oxides may include iron oxide, manganese oxide, or gadolinium oxide. Additional examples may include magnetite, maghemite, or a combination thereof. In some embodiments, the metal ion may be a carbon ion or a fluorine ion.

In an aspect, IF, B12, or an analog thereof conjugated directly or indirectly to a chelating agent may incorporate a radionuclide or metal ion. Incorporation of the radionuclide or metal ion with a chelating agent, IF, B12, or an analog thereof, may be achieved by various methods common in the art of coordination chemistry. For example, when the metal is technetium-99m, the following general procedure may be used to form a technetium complex. IF, B12, or an analog thereof-chelating agent complex solution is formed initially by dissolving the complex in aqueous alcohol such as ethanol. The solution is then degassed to remove oxygen then thiol protecting groups are removed with a suitable reagent, for example, with sodium hydroxide, and then neutralized with an organic acid, such as acetic acid (pH 6.0-6.5). In the labeling step, a stoichiometric excess of sodium pertechnetate, obtained from a molybdenum generator, is added to a solution of the complex with an amount of a reducing agent such as stannous chloride sufficient to reduce technetium and heated. The labeled complex may be separated from contaminants ^{99m}TcO₄⁻ and colloidal ^{99m}TcO₂ chromatographically, for example, with a C-18 Sep Pak cartridge.

In an alternative method, labeling can be accomplished by a transchelation reaction. The technetium source is a solution of technetium complexed with labile ligands facilitating ligand exchange with the selected chelator. Suitable ligands for transchelation include glycine, tartarate, citrate, and heptagluconate. In this instance the preferred reducing reagent is sodium dithionite. It will be appreciated that the complex may be labeled using the techniques described above, or alternatively the chelator itself may be labeled and subsequently conjugated to IF, B12, or an analog thereof to form the complex; a process referred to as the “prelabeled ligand” method.

Another approach for labeling complexes of the present invention involves immobilizing the IF, B12, or an analog thereof-chelating agent complex on a solid-phase support through a linkage that is cleaved upon metal chelation. This is achieved when the chelating agent is coupled to a functional group of the support by one of the complexing atoms. Preferably, a complexing sulfur atom is coupled to the support which is functionalized with a sulfur protecting group such as maleimide.

In another embodiment, an imaging agent may be conjugated directly or indirectly to IF, B12, or an analog thereof without the use of a chelating agent. For example, the imaging agent is conjugated directly to IF, B12, or an analog thereof. Or, the imaging agent is conjugated to a linker that is conjugated to IF, B12, or an analog thereof. For example, a radioactive iodine label (e.g., ¹²²I, ¹²³I, ¹²⁴I, ¹²⁵I, or ¹³¹I) is capable of being conjugated to each D- or L-Tyr or D- or L-4-amino-Phe residue present in a peptide linker. In an embodiment, a tyrosine residue of a peptide linker may be halogenated. Halogens include fluorine, chlorine, bromine, iodine, and astatine. Such halogenated B12s or analogs thereof may be detectably labeled if the halogen is a radioisotope, such as, for example, ¹⁸F, ⁷⁵Br, ⁷⁷Br, ¹²²I, ¹²³I, ¹²⁴I, ¹²⁵I, ¹²⁹I, ¹³¹I, or ²¹¹At. Halogenated B12s or analogs thereof contain a halogen covalently bound to at least one amino acid, and preferably to D-Tyr residues present in a peptide linker.

### (c) Therapeutic Agent

In an aspect, IF, B12, or an analog thereof may be conjugated to a therapeutic agent. In an embodiment, the IF, B12, or an analog thereof may be conjugated to a therapeutic agent, such that the therapeutic agent can be selectively targeted to a cell expressing CD206. In a specific embodiment, the therapeutic agent can be selectively targeted to a liver cell or macrophage expressing CD206. The therapeutic agent may be directly conjugated to IF, B12, or an analog thereof or may be indirectly conjugated to IF, B12, or an analog thereof. In an embodiment, the therapeutic agent may be complexed with a chelating agent that is conjugated to IF, B12, or an analog thereof. In another embodiment, the therapeutic agent may be complexed with a chelating agent that is conjugated to a linker that is conjugated to IF, B12, or an analog thereof. In still another embodiment, the therapeutic agent may be conjugated to a linker that is conjugated to IF, B12, or an analog thereof. In still yet another embodiment, the therapeutic agent may be conjugated to a linker that is conjugated to a chelating agent that is complexed with an imaging agent and conjugated to IF, B12, or an analog thereof.

A “therapeutic agent” is any compound known in the art that is used in the detection, diagnosis, or treatment of a condition or disease. Such compounds may be naturally-occurring, modified, or synthetic. Non-limiting examples of therapeutic agents may include drugs, therapeutic compounds, genetic materials, metals (such as radioactive and non-radioactive isotopes), proteins, peptides, carbohydrates, lipids, steroids, nucleic acid based materials, or derivatives, analogues, or combinations thereof in their native form or derivatized with hydrophobic or charged moieties to enhance incorporation or adsorption into a cell. Such therapeutic agents may be water soluble or may be hydrophobic. Non-limiting examples of therapeutic agents may include immune-related agents, thyroid agents, respiratory products, antineoplastic agents, anti-helmintics, anti-malarials, mitotic inhibitors, hormones, anti-protozoans, anti-tuberculars, cardiovascular products, blood products, biological response modifiers, anti-fungal agents, vitamins, peptides, anti-allergic agents, anti-coagulation agents, circulatory drugs, metabolic potentiators, anti-virals, anti-anginals, antibiotics, anti-inflammatories, anti-rheumatics, narcotics, cardiac glycosides, neuromuscular blockers, sedatives, local anesthetics, general anesthetics, or radioactive or non-radioactive atoms or ions. Non-limiting examples of therapeutic agents are described below. In a specific embodiment, a therapeutic agent may be a compound used in the detection, diagnosis, or treatment of microbial infection, arthritis, and cancer. The therapeutic agent preferably reduces or interferes with the microbial infection, arthritis, and cancer. A therapeutic agent that reduces the symptoms produced by the microbial infection, arthritis, and cancer is suitable for the present disclosure. In another specific embodiment, a therapeutic agent may be a compound used in the detection, diagnosis, or treatment of a respiratory infection. In another specific embodiment, a therapeutic agent may be a compound used in the detection, diagnosis, or treatment of a viral infection. In another specific embodiment, a therapeutic agent may be a compound used in the detection, diagnosis, or treatment of a viral respiratory infection.

IF, B12, or an analog thereof may be conjugated to one, two, three, four, or five therapeutic agents. A linker may or may not be used to conjugate a therapeutic agent to IF, B12, or an analog thereof. Generally speaking, the conjugation should not interfere with intrinsic factor binding to B12 or an analog thereof. Additionally, the conjugation should not interfere with IF binding to CD206. In some instances, IF, B12, or an analog thereof may be generated with a cleavable linkage between the IF, B12, or analog thereof and therapeutic agent. Such a linker may allow release of the therapeutic agent at a specific cellular location.

A therapeutic agent of the invention may be a small molecule therapeutic, a therapeutic antibody, a therapeutic nucleic acid, a therapeutic protein or peptide, or a chemotherapeutic agent. Non-limiting examples of therapeutic antibodies may include muromomab, abciximab, rituximab, daclizumab, basiliximab, palivizumab, infliximab, trastuzumab, etanercept, gemtuzumab, alemtuzumab, ibritomomab, adalimumab, alefacept, omalizumab, tositumomab, efalizumab, cetuximab, bevacizumab, natalizumab, ranibizumab, panitumumab, eculizumab, and certolizumab. A representative therapeutic nucleic acid may encode a therapeutic protein or peptide, including but not limited to a polypeptide having an ability to induce an immune response and/or an anti-angiogenic response in vivo. Alternatively, a therapeutic nucleic acid may be a single-stranded nucleic acid or double-stranded nucleic acid that is able to interfere with gene expression in a targeted, sequence-based manner. The nucleic acid may comprise ribonucleotides, modified ribonucleotides, deoxynucleotides, deoxyribonucleotides, or nucleotide analogues. Representative therapeutic proteins with immunostimulatory effects include but are not limited to cytokines (e.g., an interleukin (IL) such as IL2, IL4, IL7, IL12, interferons, granulocyte-macrophage colony-stimulating factor (GM-CSF), tumor necrosis factor alpha (TNF-α)), immunomodulatory cell surface proteins (e.g., human leukocyte antigen (HLA proteins), co-stimulatory molecules, and tumor-associated antigens. See Kirk & Mule, 2000; Mackensen et al., 1997; Walther & Stein, 1999; and references cited therein. Representative therapeutic proteins with anti-angiogenic activities that can be used in accordance with the presently disclosed subject matter include: thrombospondin I (Kosfeld & Frazier, 1993; Tolsma et al., 1993; Dameron et al., 1994), metallospondin proteins (Carpizo & Iruela-Arispe, 2000), class I interferons (Albini et al., 2000), IL12 (Voest et al., 1995), protamine (Ingber et al., 1990), angiostatin (O'Reilly et al., 1994), laminin (Sakamoto et al., 1991), endostatin (O'Reilly et al., 1997), and a prolactin fragment (Clapp et al., 1993). In addition, several anti-angiogenic peptides have been isolated from these proteins (Maione et al., 1990; Eijan et al., 1991; Woltering et al., 1991). Representative proteins with both immunostimulatory and anti-angiogenic activities may include IL12, interferon-γ, or a chemokine. Other therapeutic nucleic acids that may be useful for cancer therapy include but are not limited to nucleic acid sequences encoding tumor suppressor gene products/antigens, antimetabolites, suicide gene products, and combinations thereof.

A chemotherapeutic agent refers to a chemical compound that is useful in the treatment of cancer. The compound may be a cytotoxic agent that affects rapidly dividing cells in general, or it may be a targeted therapeutic agent that affects the deregulated proteins of cancer cells. A cytotoxic agent is any naturally-occurring, modified, or synthetic compound that is toxic to tumor cells. Such agents are useful in the treatment of neoplasms, and in the treatment of other symptoms or diseases characterized by cell proliferation or a hyperactive cell population. The chemotherapeutic agent may be an alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof. In an exemplary embodiment, the chemotherapeutic agent is selected from the group consisting of liposomal doxorubicin and nanoparticle albumin docetaxel.

Non-limiting examples of suitable alkylating agents may include altretamine, benzodopa, busulfan, carboplatin, carboquone, carmustine (BCNU), chlorambucil, chlornaphazine, cholophosphamide, chlorozotocin, cisplatin, cyclosphosphamide, dacarbazine (DTIC), estramustine, fotemustine, ifosfamide, improsulfan, lipoplatin, lomustine (CCNU), mafosfamide, mannosulfan, mechlorethamine, mechlorethamine oxide hydrochloride, melphalan, meturedopa, mustine (mechlorethamine), mitobronitol, nimustine, novembichin, oxaliplatin, phenesterine, piposulfan, prednimustine, ranimustine, satraplatin, semustine, temozolomide, thiotepa, treosulfan, triaziquone, triethylenemelamine, triethylenephosphoramide (TEPA), triethylenethiophosphaoramide (thiotepa), trimethylolomelamine, trofosfamide, uracil mustard and uredopa.

Suitable anti-metabolites may include, but are not limited to aminopterin, ancitabine, azacitidine, 8-azaguanine, 6-azauridine, capecitabine, carmofur (1-hexylcarbomoyl-5-fluorouracil), cladribine, clofarabine, cytarabine (cytosine arabinoside (Ara-C)), decitabine, denopterin, dideoxyuridine, doxifluridine, enocitabine, floxuridine, fludarabine, 5-fluorouracil, gemcitabine, hydroxyurea (hydroxycarbamide), leucovorin (folinic acid), 6-mercaptopurine, methotrexate, nafoxidine, nelarabine, oblimersen, pemetrexed, pteropterin, raltitrexed, tegofur, tiazofurin, thiamiprine, tioguanine (thioguanine), and trimetrexate.

Non-limiting examples of suitable anti-tumor antibiotics may include aclacinomysin, aclarubicin, actinomycins, adriamycin, aurostatin (for example, monomethyl auristatin E), authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, epoxomicin, esorubicin, idarubicin, marcellomycin, mitomycins, mithramycin, mycophenolic acid, nogalamycin, olivomycins, peplomycin, plicamycin, potfiromycin, puromycin, quelamycin, rodorubicin, sparsomycin, streptonigrin, streptozocin, tubercidin, valrubicin, ubenimex, zinostatin, and zorubicin.

Non-limiting examples of suitable anti-cytoskeletal agents may include cabazitaxel, colchicines, demecolcine, docetaxel, epothilones, ixabepilone, macromycin, omacetaxine mepesuccinate, ortataxel, paclitaxel (for example, DHA-paclitaxel), taxane, tesetaxel, vinblastine, vincristine, vindesine, and vinorelbine.

Suitable topoisomerase inhibitors may include, but are not limited to, amsacrine, etoposide (VP-16), irinotecan, mitoxantrone, RFS 2000, teniposide, and topotecan.

Non-limiting examples of suitable anti-hormonal agents may include aminoglutethimide, antiestrogen, aromatase inhibiting 4(5)-imidazoles, bicalutamide, finasteride, flutamide, fluvestrant, goserelin, 4-hydroxytamoxifen, keoxifene, leuprolide, LY117018, mitotane, nilutamide, onapristone, raloxifene, tamoxifen, toremifene, and trilostane.

Examples of targeted therapeutic agents may include, without limit, monoclonal antibodies such as alemtuzumab, cartumaxomab, edrecolomab, epratuzumab, gemtuzumab, gemtuzumab ozogamicin, glembatumumab vedotin, ibritumomab tiuxetan, reditux, rituximab, tositumomab, and trastuzumab; protein kinase inhibitors such as bevacizumab, cetuximab, crizonib, dasatinib, erlotinib, gefitinib, imatinib, lapatinib, mubritinib, nilotinib, panitumumab, pazopanib, sorafenib, sunitinib, toceranib, and vandetanib.

Non limiting examples of angiogeneisis inhibitors may include angiostatin, bevacizumab, denileukin diftitox, endostatin, everolimus, genistein, interferon alpha, interleukin-2, interleukin-12, pazopanib, pegaptanib, ranibizumab, rapamycin (sirolimus), temsirolimus, and thalidomide.

Non limiting examples of growth inhibitory polypeptides may include bortazomib, erythropoietin, interleukins (e.g., IL-1, IL-2, IL-3, IL-6), leukemia inhibitory factor, interferons, romidepsin, thrombopoietin, TNF-α, CD30 ligand, 4-1BB ligand, and Apo-1 ligand.

Non-limiting examples of photodynamic therapeutic agents may include aminolevulinic acid, methyl aminolevulinate, retinoids (alitretinon, tamibarotene, tretinoin), and temoporfin.

Other antineoplastic agents may include anagrelide, arsenic trioxide, asparaginase, bexarotene, bropirimine, celecoxib, chemically linked Fab, efaproxiral, etoglucid, ferruginol, lonidamide, masoprocol, miltefosine, mitoguazone, talapanel, trabectedin, and vorinostat.

Non-limiting examples of antibiotics may include penicillins, tetracyclines, cephalosporins, quinolones, lincomycins, macrolides, sulfonamides, glycopeptides, aminoglycosides, and carbapenems. Non-limiting examples of specific antibiotics may include amoxicillin, doxycycline, cephalexin, ciprofloxacin, clindamycin, metronidazole, azithromycin, sulfamethoxazole/trimethoprim, amoxicillin/clavulanate, and levofloxacin.

Non-limiting examples of anti-inflammatories may include chloroquine, diclofenac, etodolac, fenoprofen, flurbiprofen, hydroxychloroquine, ibuprofen, indomethacin, meclofenamate, mefenamic acid, meloxicam, nabumetone, naproxen, oxaprozin, piroxicam, sulindac, tolmetin, and celecoxib.

Non-limiting examples of anti-viral agents include ACE inihibitors, protease inhibitors, nucleoside analogs, polymerase inhibitors, integrase inhibitors, fusion inihibors, ribozymes, TLR4 antagonists, IL-6 receptor antagonists, and neuraminidase inhibitors. Non-limiting examples of specific anti-viral agents include abacavir, acyclovir, adefovir, amantadine, ampligen, amprenavir, arbidol, atazanavir, atripla, balavir, baloxavir marboxil, baricitinib, biktavy, boceprevir, cidofovir, clevudine, cobicistat, combivir, daclatasvir, darunavir, delavirdine, descovy, didaanosine, docosanol, dolutegravir, doravirine, ecoliever, deoxudine, efavirenz, elvitegravir, emtricitabine, enfuvirtide, entecavir, etravirine, famciclovir, favipiravir, fluoxetine, fludase (DAS181), fluvoxamine, fomivirsen, fosamprenavir, foscarnet, fosfonet, ganciclovir, ibacitabine, ibalizumab, idoxuridine, imiquimod, imunovir, indinavir, inosine, interferon type I, interferon type II, interferon type III, lamivudine, letermovir, lopinavir, loviride, maraviroc, methisazone, moroxydine, nelfinavir, nevirapine,nexavir, nitazoxanide, norvir, oseltamivir, peginterferon alfa-2a, peginterferon alfa-2b, peginterferon lambda, penciclovir, peramivir, pleconaril, podophyllotoxin, pyramidine, raltegravir, REGN-EB3, remdesivir, ribavirin, rilpivirine, rimantadine, ritonavir, ruxolitinib, sarilumab, saquinavir, simeprevir, sofosbuvir, stavudine, telaprevir, telbivudine, tenofovir alafenamide, tenofovir disoproxil, tenofovir, tipranavir, trifluiridine, trizivir, tromantadine, truvada, umifenovir, valaciclovir, valganciclovir, vicriviroc, vidarabine, viramidine, zalcitabine, zanamivir, and zidovudine.

Also included are pharmaceutically acceptable salts, acids, or derivatives of any of the above listed therapeutic agents. Generally speaking, a derivative of therapeutic agent refers to a therapeutic agent that has been modified so as to contain a functional group that is reactive (e.g., a nucleophile, an electrophile, etc.). In some instances, a derivative may be a therapeutic agent reduced back to free a functional group. A non-limiting example is a compound of formula (II) wherein R₂ is an amino group and R₃ is a methyl group, which is a chloroquine derivative. In other instances, a derivative may have a functional group that cannot be derived from the therapeutic agent. As a non-limiting example, a compound of formula (II) wherein R₂ is a thiocyanato group and R₃ is a methyl group is also a chloroquine derivative.

As used herein, the term “chloroquine derivative” encompasses compounds of formula I:

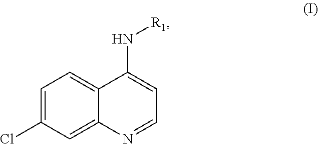

wherein R₁ is a linker capable of attaching to B12, B12 analog or IF. In some embodiments, R₁ is selected from the group consisting of hydrogen, alkyl, substituted alkyl, alkenyl, substituted alkenyl, alkynl, substituted alkynl, aryl, substituted aryl, carbocyclo, and heterocyclo. In further embodiments, R₁ is selected from the group consisting of C₂-C₁₀ substituted alkyl, C₂-C₁₀ alkenyl, C₂-C₁₀ substituted alkenyl, C₂-C₁₀ alknyl, and C₂-C₁₀ substituted alkynl. In still further embodiments, a compound of formula (I) is a compound of formula (II):

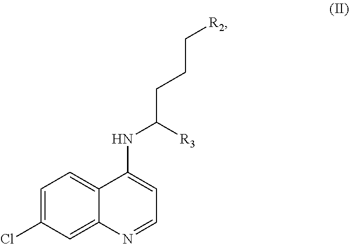

wherein R₂ comprises a functional group capable of attaching to B12, B12 analog, or IF, and R₃ is selected from the group consisting of hydrogen, alkyl, substituted alkyl, alkenyl, substituted alkenyl, alkynl, substituted alkynl, aryl, substituted aryl, carbocyclo, and heterocyclo. In some embodiments, R₂ is selected from the group consisting of substituted hydrocarbyl, alkyl, alkoxy, acyl, acyloxy, alkenyl, alkenoxy, aryloxy, amino, amido, acetal, carbamyl, carbocyclo, cyano, ester, ether, halo, heterocyclo, hydroxyl, keto, ketal, phospho, nitro, thiocyanato, and thio. In still further embodiments, a compound of formula (I) is a compound of formula (III):

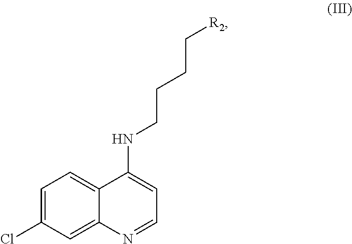

wherein R₂ is as defined above. In a specific embodiment, a compound of formula (I) is a compound of formula (II):

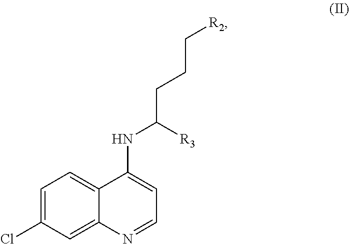

wherein R₂ is an amino group and R₃ is hydrogen, C₁-C₈ alkyl, or C₁-C₈ substituted alkyl. In another specific embodiment, a compound of formula (I) is a compound of formula (II):

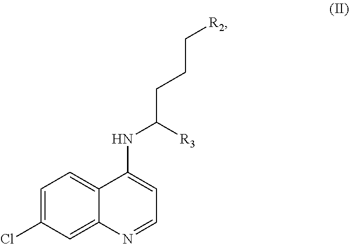

wherein R₂ is an amino group and R₃ is hydrogen, C₁-C₄ alkyl, or C₁-C₄ substituted alkyl. In another specific embodiment, a compound of formula (I) is a compound of formula (II):

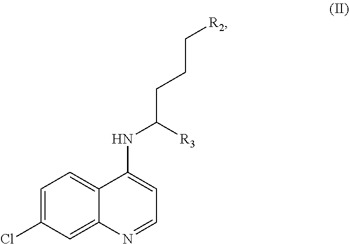

wherein R₂ is an amino group and R₃ is hydrogen or methyl. In another specific embodiment, a compound of formula (I) is a compound of formula (III):

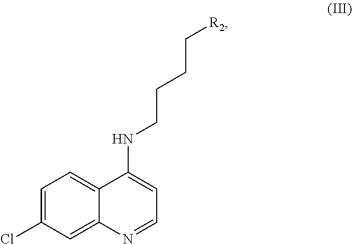

wherein R₂ is an amino group.

The dose of the therapeutic agent can and will vary. For instance, the dose of a chemotherapeutic agent can and will vary depending upon the agent and the type of tumor or neoplasm. A skilled practitioner will be able to determine the appropriate dose of the chemotherapeutic agent or other therapeutic agent.

Other therapeutic agents may comprise a virus or a viral genome such as an oncolytic virus. An oncolytic virus comprises a naturally occurring virus that is capable of killing a cell in the target tissue (for example, by lysis) when it enters such a cell.

### (d) Intrinsic Factor

Intrinsic factor (IF) is a glycosylated protein that is secreted from the gastric mucosa and the pancreas. IF binds B12 with picomolar affinity (K_{d}˜ 1 pM). In the B12 uptake pathway, the IF protein facilitates transport of B12 across the intestinal enterocyte, which occurs by receptor-mediated endocytosis at the apically expressed IF-B12 receptor (cubilin; CUBN). CUBN works to transport B12 in concert with an anchoring protein amnionless (Am). Following transcytosis, and between 2.5 and 4 h after initial ingestion, B12 appears in blood plasma bound to the third trafficking protein, transcobalamin (TC). Cells that require B12 express the holo-TC receptor, CD320. Upon internalization, TC is degraded and B12 is transported from the lysosome for cellular use.

As IF binds B12 with picomolar affinity, production of recombinant IF in the presence of B12 results in IF pre-bound with B12 (holo-IF). To achieve apo-IF, IF may be expressed and purified from a transgenic plant. Plants do not utilize B12, minimizing holo-IF production. In an embodiment, IF may be expressed and purified from Arabidopsis. More specifically, IF may be expressed and purified from Arabidopsis thaliana. As IF of the disclosure is expressed and purified from a transgenic plant, the IF has a specific glycosylation pattern that differs from IF produced in humans. In an embodiment, IF is glycosylated with a(1-3)-fucose, xylose, mannose and n-acetylglucosamine. More specifically IF is glycosylated with a(1-3)-fucose, xylose, mannose and n-acetylglucosamine in ratios of 0.17:0.18:1.0:0.24, respectively. Any transgenic plant may be used as a source of IF, provided expression and purification from the plant results in IF with fucose, mannose, or GlcNac terminal moieties, preferably in ratios of about 0.17: about 0.18: about 1.0: about 0.24. For instance, in another embodiment, IF may be expressed and purified from Nicotiana. More specifically, IF may be expressed and purified from Nicotiana benthamiana.

The unique glycosylation pattern of IF produced in plants surprisingly changes the receptor specificity of IF. In an embodiment, IF of the disclosure binds to the asialoglycoprotein receptor (ASGPR) or CD206 receptor (MR; MCRC1). In a specific embodiment, IF of the disclosure binds to the CD206 receptor. In some embodiments, IF of the disclosure binds to both CD206 and Cubilin, which is the canonical receptor for IF. CD206 is a member of the C-type lectin superfamily and is produced by most tissues macrophages and select endothelial and dendritic cells and plays a key role in the innate and adaptive immune response in humans. Tumor-associated macrophages (TAM) positive for CD206 have been shown to contribute to tumor growth, metastasis, and relapse. CD206 has also been shown to be involved in leukocyte trafficking and inflammation. Accordingly, targeting of CD206 may be used to image, diagnose and/or treat disease including microbial infection, arthritis, and cancer. In another embodiment, IF of the disclosure binds to liver cells and macrophages through the CD206 receptor.

IF of the disclosure may be expressed and purified via standard methodology. The expressed and purified IF may be from any species, provided it binds to B12 or a B12 conjugate. In a specific embodiment, the IF is recombinant human IF. A skilled artisan will appreciate that IF can be found in a variety of species. Non-limiting examples include human (NP_005133.2), mouse (P52787.2), rat (NP_058858.1), dog (Q5XWD5.1), cat (XP_003993466.1), cattle (NP_001193168.1), non-human primates (EHH56203.1, XP_004051305.1), and horse (XP_008508117.1). It is appreciated that the present invention is directed to homologs of IF in other organisms and is not limited to the human protein. Homologs can be found in other species by methods known in the art. For example, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www.ncbi.nlm.nih.gov for more details. In some embodiments, a homolog has at least 80%, at least 81%, at least 82%, at least 83%, at least 84%, at least 85%, at least 86%, at least 87%, at least 88%, or 89% identity to human IF. In another embodiment %, a homolog has at least 90%, at least 91 at least %, at least 92 at least %, at least 93 at least %, at least 94%, at least 95%, at least 96%, at least 97%, at least 98%, at least 99%, or at least 100% identity to human IF. For instance, a homolog may have at least 80%, at least 81%, at least 82%, at least 83%, at least 84%, at least 85%, at least 86%, at least 87%, at least 88%, or 89% identity to human IF. In another embodiment %, a homolog has at least 90%, at least 91 at least %, at least 92 at least %, at least 93 at least %, at least 94%, at least 95%, at least 96%, at least 97%, at least 98%, at least 99%, or at least 100% identity to the IF sequence accession number NP_005133.2.

In a specific embodiment, the IF comprises the sequence disclosed in accession number NP_005133.2. In other embodiments, the IF comprises the sequence disclosed in accession number NP_005133.2 but for one to 10 conservative amino acid substitutions. For example, the IF comprises the sequence disclosed in accession number NP_005133.2 but for 1, 2, 3, 4, 5, 6, 7, 8, 9 or 10 conservative amino acid substitutions. As used herein, a “conservative amino acid substitution” is one in which the amino acid residue is replaces with an amino acid residue having a similar side chain. Families of amino acid residues having similar side chains have been defined in the art. These families include amino acids with basic side chains (e.g. lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g. glycine, alanine, valine, leucine, isoleucine, proline, phenylalanine, methionine, tryptophan, histidine). The resulting peptide comprising the substitution should have similar characteristics or properties including size, hydrophobicity, etc., such that the overall functionally of the peptide does not significantly change. As the structure of IF bound to B12 is known in the art, a skilled artisan would be able to determine amino acids essential to B12 binding to ensure binding to B12 or a B12 conjugate.

In an aspect, IF is bound to B12 or to a B12 conjugate of the disclosure thereby forming a complex. Importantly, IF pre-bound to B12 or a B12 conjugate is not affected by endogenous B12 levels when administered to a subject. Accordingly, B12 or B12 conjugates pre-bound to IF overcomes a concern of interference with functional B12 levels. The IF may be bound to B12 or analog thereof before or after conjugation to an imaging, diagnostic, or therapeutic agent. In a specific embodiment, IF may be bound to B12 or an analog thereof after conjugation to an imaging, diagnostic, or therapeutic agent. In an embodiment, IF (alone or conjugated) may be pre-bound to a B12 or B12 conjugate by combining B12 with IF in solution. By way of non-limiting example, B12 or B12 conjugate may be combined with IF or IF conjugate in PBS at pH 7.4 or in MES buffer at pH 5.5 or in water at pH 8 at temperatures ranging from about 25° C. to about 37° C. For binding, IF or IF conjugate may be contacted with B12 or B12 conjugate for at least 30 minutes. Alternatively, IF or IF conjugate may be contacted with B12 or B12 conjugate for at least 15 minutes, at least 30 minutes, at least 45 minutes, at least 1 hour, at least 2 hours, at least 3 hours, at least 4 hours, at least 5 hours or at least 6 hours. A skilled artisan would be able to determine the various conditions upon which IF or IF conjugate and B12 or B12 conjugate may be pre-bound.

For pre-binding of IF or IF conjugate and B12 or the B12 conjugate, IF or IF conjugate and B12 or the B12 conjugate may be combined in solution. One IF or IF conjugate binds to one B12 or B12 conjugate. Accordingly, the ratio of IF or IF conjugate to B12 or B12 conjugate added to solution may be 1:1. However, to facilitate saturation of the IF or IF conjugate with B12 or B12 conjugate, a greater amount of IF or IF conjugate may be added to solution relative to B12 or B12 conjugate. For example, the ratio of IF or IF conjugate to B12 or B12 conjugate may be 1.1:1, 1.2:1, 1.3:1, 1.4:1, 1.5:1, 2:1, 2.5:1, 3:1, 3.5:1, 4:1, 4.5:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, or 100:1. In specific embodiments, the ratio of IF or IF conjugate to B12 or B12 conjugate may be 1:0.9, 1:0.8, 1:0.7, 1:0.6, 1:0.5, 1:0.4, 1:0.3, 1:0.2, or 1:0.1. In an exemplary embodiment, the ratio of IF or IF conjugate to B12 or B12 conjugate is 1:0.8. In other embodiments, an excess of 5% or more IF or IF conjugate relative to B12 or B12 conjugate may be added to solution. For example, an excess of 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 70%, 80%, 90% or 100% IF or IF conjugate relative to B12 or B12 conjugate may be added to solution. In specific embodiments, an excess of 5%, 6%, 7%, 8%, 9%, 10%, 11%, 12%, 13%, 14%, or 15% IF or IF conjugate relative to B12 or B12 conjugate may be added to solution. Preferably, in some embodiments, excess IF or IF conjugate is added to the solution relative to B12 or B12 conjugate. However, it may be necessary to add a greater amount of B12 or B12 conjugate relative to IF or IF conjugate to reduce or eliminate unbound IF. Accordingly, the ratio of B12 or B12 conjugate to IF or IF conjugate may be 1.1:1, 1.2:1, 1.3:1, 1.4:1, 1.5:1, 2:1, 2.5:1, 3:1, 3.5:1, 4:1, 4.5:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, or 100:1. In specific embodiments, the ratio of B12 or B12 conjugate to IF or IF conjugate may be 1.1:1, 1.2:1, 1.3:1, 1.4:1, 1.5:1, 2:1, 2.5:1, 3:1, 3.5:1, 4:1, 4.5:1, 5:1, 6:1, 7:1, 8:1, 9:1, or 10:1. In other embodiments, an excess of 5% or more B12 or B12 conjugate relative to IF or IF conjugate may be added to solution. For example, an excess of 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 70%, 80%, 90% or 100% B12 or B12 conjugate relative to IF or IF conjugate may be added to a solution. In a specific embodiment, an excess of 5%, 6%, 7%, 8%, 9%, 10%, 11%, 12%, 13%, 14%, or 15% B12 or B12 conjugate relative to IF or IF conjugate may be added to a solution. Prior to administration of a pharmaceutical formulation of the disclosure, it may be necessary to remove unbound IF or IF conjugate and/or unbound B12 or B12 conjugate. In the case of imaging, removal of unbound B12 or B12 conjugate may be necessary to reduce background.

Conjugation of IF to a therapeutic, diagnostic, or imaging agent may be via solvent exposed amino acids such as, but not limited to, lysine, cysteine, aspartic acid, or glutamic acid.

### (e) Pharmaceutical Formulation

The present disclosure also provides pharmaceutical formulations for parenteral, oral, and topical administration, including administration via inhalation. The pharmaceutical formulation comprises (a) recombinantly produced intrinsic factor (IF) with a glycosylation pattern that enables binding to CD206, optional B12 or B12 analog, and at least one therapeutic, diagnostic, or imaging agent; wherein the IF is conjugated to a therapeutic, diagnostic, or imaging agent; or (b) recombinantly produced intrinsic factor (IF) with a glycosylation pattern that enables binding to CD206, B12 or B12 analog, and at least one therapeutic, diagnostic, or imaging agent; wherein the B12 or B12 analog is conjugated to a therapeutic, diagnostic, or imaging agent; and further comprises at least one pharmaceutically acceptable carrier for parenteral, oral, or topical administration, including administration via inhalation. The term parenteral as used herein includes subcutaneous, intravenous, intramuscular, intradermal, intra-arterial, intraosseous, intraperitoneal, or intrathecal injection, or infusion techniques. In one embodiment, the disclosure encompasses a formulation for IV administration, the formulation comprising intrinisic factor and B12 or a B12 conjugate, as an active ingredient, and at least one pharmaceutically acceptable carrier for IV administration. In one embodiment, the disclosure encompasses a formulation for inhalation, the formulation comprising intrinisic factor and B12 or a B12 conjugate, as an active ingredient, and at least one pharmaceutically acceptable carrier for inhalation.

The composition can be formulated into various dosage forms and administered by a number of different means that will deliver a therapeutically effective amount of the active ingredient. Such compositions can be administered parenterally, orally, or topically in dosage unit formulations containing conventional nontoxic pharmaceutically acceptable carriers, adjuvants, and vehicles as desired. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L., Eds., Pharmaceutical Dosage Forms, Marcel Dekker Inc., New York, N.Y. (1980).

For parenteral administration, the preparation may be an aqueous or an oil-based solution. Aqueous solutions may include a sterile diluent or excipient such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

In certain embodiments, a composition comprising an IF conjugate or B12 conjugate is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of the IF conjugate or B12 conjugate in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the IF conjugate or B12 conjugate may be selectively delivered to a cell by encapsulation in a liposome that fuses with the targeted cell's membrane.

Liposomes may be comprised of a variety of different types of phospolipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying an IF conjugate or a B12 conjugate (e.g., having at least one methionine compound) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The IF and B12 conjugate may be encapsulated in a microemulsion by any method generally known in the art.

In yet another embodiment, an IF conjugate may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

In an additional aspect, a composition of the invention may be administered via inhalation. Inhalation of a composition of the invention results in administration directly to one or more desired regions of the respiratory tract, which includes the upper respiratory tract (e.g., nasal, sinus, and pharyngeal compartments), the respiratory airways (e.g., laryngeal, tracheal, and bronchial compartments) and the lungs or pulmonary compartments (e.g., respiratory bronchioles, alveolar ducts, alveoli, alveoli-capillary barrier). Alveolar macrophages are exquisitely sensitive to their local environment. A composition of the present disclosure may bind to CD206 receptors on alveolar macrophages and other cell types in the lungs and serve as a carrier of diagnostic or therapeutic agents. Inhalation may be effected in certain preferred embodiments through intra-nasal or oral inhalation. For instance, a composition of the invention may be formulated with an e-liquid carrier to be delivered via a vaping device, or as a dry powder to be delivered via a dry powder inhaler, or with a propellant to be delivered by a metered-dose inhaler, or with a liquid or gaseous carrier to be delivered as a nasal spray, directly to the alveolar epithelium and thereby modify the inflammatory, infective, fibrosing, or neoplastic condition affecting the lung tissue. In another example, a composition of the invention may be formulated for inhalation (e.g. vaping, inhaler, nasal spray, nebulizer, atomizer, etc.) to modulate a biofilm, mucous, or protein exudate in the lungs/respiratory tract. In another example, a composition of the invention may be formulated for inhalation (e.g. vaping, inhaler, nasal spray, nebulizer, atomizer, etc.) to modulate bronchodilation, for treatment of respiratory issues such as asthma. This ‘topical’ delivery can provide precision dosing with mitigation of systemic side effects.

### II. Methods

The present disclosure further encompasses a method of delivering a therapeutic, diagnostic, or imaging agent to a cell expressing CD206 in a subject, the method comprising: administering to the subject a pharmaceutical formulation as detailed above comprising (i) IF conjugated to a therapeutic, diagnostic, or imaging agent, or (ii) B12 or B12 analog conjugated to a therapeutic, diagnostic, or imaging agent, and IF; wherein the IF binds to CD206 in the subject thereby delivering the therapeutic, diagnostic, or imaging agent. In some embodiments, the CD206 is expressed on a liver cell of the subject. In some embodiments, the CD206 is expressed on a macrophage and/or an immature dendritic cell of the subject. In some embodiments, the CD206 is expressed on an alveolar macrophage and/or an immature dendritic cell of the subject.

In another aspect, a pharmaceutical formulation of the present disclosure, as described above, may be used in treating, stabilizing and preventing microbial infection, inflammation, fibrosis, lung disease or cancer in a subject, the method comprising: administering to the subject a pharmaceutical formulation as detailed above comprising (i) IF conjugated to a therapeutic agent, or (ii) B12 or B12 analog conjugated to a therapeutic agent, and IF.

By “treating, stabilizing, or preventing microbial infection” is meant causing a reduction in the presence of bacteria, fungi, parasites and/or virus in a subject. A reduction in presence of bacteria, fungi, parasites and/or virus may be measured by alleviation of symptoms and/or reduction of fever. In some embodiments, a microbial infection may be a respiratory infection.

By “treating, stabilizing, or preventing inflammation” is meant causing a reduction in inflammation in a subject. A reduction in inflammation may be measured by alleviation of symptoms and/or reduction of tenderness, pain, swelling and/or redness. Inflammation, as used herein, may encompass such diseases or disorders such as nonalcoholic steatohepatitis (NASH) or hepatitis. Other methods of the present disclosure may include treating, stabilizing, or preventing psoriasis, arthritis, autoimmune disorders, and primary biliary cirrhosis.

By “treating, stabilizing, or preventing fibrosis” is meant causing a reduction in fibrosis. A reduction in fibrosis may be measured via imagining, or by monitoring symptoms. In some embodiments, a pharmaceutical formulation of the present disclosure may be used to treat, stabilize or prevent liver fibrosis. In some embodiments, a pharmaceutical formulation of the present disclosure may be used to treat, stabilize or prevent pulmonary fibrosis.

Still other methods of the present disclosure may include treating, stabilizing or preventing lung disease including but not limited to asthma, chronic obstructive pulmonary disease (COPD), pulmonary fibrosis, idiopathic pulmonary fibrosis, radiation induced fibrosis, silicosis, asbestos induced pulmonary or pleural fibrosis, acute lung injury, acute respiratory distress syndrome (ARDS), sarcoidosis, usual interstitial pneumonia (UIP), cystic fibrosis, Chronic lymphocytic leukemia (CLL)-associated fibrosis, Hamman-Rich syndrome, Caplan syndrome, coal worker's pneumoconiosis, cryptogenic fibrosing alveolitis, obliterative bronchiolitis, chronic bronchitis, emphysema, pneumonitis, Wegner's granulamatosis, lung scleroderma, silicosis, interstitial lung disease, asbestos induced pulmonary and/or pleural fibrosis. By “treating, stabilizing, or preventing lung disease” is meant causing a reduction in one or more symptoms or signs of lung disease, causing a reduction in the number of days hospitalized and/or the number of days in an intensive care unit, causing a reduction in the days of mechanical intervention, or causing a reduction in the need for some other medical or surgical intervention. Symptoms or signs of lung disease include but not limited to pulmonary fibrosis, inflammation, pulmonary coagulopathy, scarring of lung tissue, shortness of breath, loss of functional alveoli, airway hyperreactivity, etc.

By “treating, stabilizing, or preventing cancer” is meant causing a reduction in the size of a tumor or in the number of cancer cells, slowing or preventing an increase in the size of a tumor or cancer cell proliferation, increasing the disease-free survival time between the disappearance of a tumor or other cancer and its reappearance, preventing an initial or subsequent occurrence of a tumor or other cancer, or reducing an adverse symptom associated with a tumor or other cancer. In a desired embodiment, the percent of tumor or cancerous cells surviving the treatment is at least 20, 40, 60, 80, or 100% lower than the initial number of tumor or cancerous cells, as measured using any standard assay (e.g., caspase assays, TUNEL and DNA fragmentation assays, cell permeability assays, and Annexin V assays). Desirably, the decrease in the number of tumor or cancerous cells induced by administration of a composition of the invention is at least 2, 5, 10, 20, or 50-fold greater than the decrease in the number of non-tumor or non-cancerous cells. Desirably, the methods of the present invention result in a decrease of 20, 40, 60, 80, or 100% in the size of a tumor or in the number of cancerous cells, as determined using standard methods. Desirably, at least 20, 40, 60, 80, 90, or 95% of the treated subjects have a complete remission in which all evidence of the tumor or cancer disappears. Desirably, the tumor or cancer does not reappear or reappears after at least 5, 10, 15, or 20 years.

The pharmaceutical formulation of the present disclosure may be part of a combination therapy. Preferably, a combination therapy would include the use of the pharmaceutical formulation of the present disclosure along with a radiation therapy or chemotherapy course of treatment in embodiments directed to cancer. In embodiments directed to treating a microbial infection, a combination therapy may include the use of the pharmaceutical formulation of the present disclosure along with a vaccine, an anti-inflammatory agent, etc. In embodiments directed to treating a respiratory infection, a combination therapy may include the use of the pharmaceutical formulation of the present disclosure administered by inhalation along with pharmaceutical formulation comprising the same or different drug administered orally or parenterally.

In yet another aspect, the present disclosure provides a method of detecting a microbial infection, lung disease, fibrosis, inflammation, arthritis, or cancer in a subject. The method comprises administering to the subject a pharmaceutical formulation comprising (i) IF conjugated to an imaging agent, or (ii) B12 or B12 analog conjugated to an imaging agent, and IF; and detecting the imaging agent, wherein the presence of the imaging agent indicates the presence of microbial infection, lung disease, fibrosis, inflammation, arthritis, or cancer in the subject. In a specific embodiment, the IF binds to CD206. In another specific embodiment, the IF binds to CD206 expressed on liver cells, macrophages, immature dendritic cells, or any combination thereof. In preferred embodiments, the methods may be used to diagnose or image a microbial infection, arthritis or cancer in a subject. In other embodiments, the methods may be used to image CD206 expression in a subject. In some embodiments, a method for detecting cancer can comprise (a) biopsying a suspected tumor; (c) contacting a pharmaceutical formulation of the disclosure with the suspected tumor in vitro; and (d) detecting the imaging agent in a tissue, whereby a tumor is diagnosed.

Binding may be detected using microscopy (fluorescent microscopy, confocal microscopy, or electron microscopy), magnetic resonance imaging (including MTI, MRS, DWI and fMRI), scintigraphic imaging (SPECT (Single Photon Emission Computed Tomography), PET (Positron Emission Tomography), gamma camera imaging, and rectilinear scanning), radiography, or ultrasound. The imaging agent may be detectable in situ, in vivo, ex vivo, and in vitro.

In still yet another aspect, the present disclosure provides a method of delivering an agent to a cell that expresses CD206 in a subject. The method comprises administering a complex of recombinantly produced IF, optional B12 or B12 analog and the agent as detailed herein to the subject. Accordingly, the complex may bind to CD206 present on a cell thereby delivering the agent to the cell. In an embodiment, the pharmaceutical composition comprises an imaging, diagnostic, or therapeutic agent. Such a method may be used to detect or treat a cell that expresses CD206 in a subject.

In yet still another aspect, the present disclosure provides a method of modulating CD206 function. The method comprises administering a pharmaceutical composition detailed above to the subject. Accordingly, the complex of IF may bind to CD206 present on a cell thereby modulating CD206 function. By modulate is meant to change the activity of CD206. For example, the complex may block CD206 function thereby inhibiting the activity of CD206. As CD206 has been shown to contribute to tumor growth, metastasis, and relapse, inhibiting the activity of CD206 may reduce tumor growth, metastasis, and relapse. Additionally, as CD206 has been shown to be involved in leukocyte trafficking and inflammation, inhibiting the activity of CD206 may reduce leukocyte trafficking and inflammation.

In each of the above aspects and embodiments, the IF, B12, or B12 analog of the pharmaceutical formulation may be indirectly or directly conjugated to imaging agent(s) and/or therapeutic agent(s) as described in Section I in order to provide specific delivery of a diagnostic, imaging agent, or therapy to the site of microbial infection, lung disease, fibrosis, inflammation, arthritis, or cancer. For example, in embodiments where IF is conjugated to imaging agent(s) and/or therapeutic agent(s), the IF conjugate administered binds CD206. Alternatively, in embodiments where B12 or B12 analog is conjugated to imaging agent(s) and/or therapeutic agent(s), an IF complex comprising the B12/B12 analog conjugate binds CD206. As detailed in Section I, the IF and B12/B12 analog may be pre-bound to form complex (i.e., the complex is part of the pharmaceutical formulation) or formed in vivo following administration. The IF conjugate or IF complex is then internalized and the imaging agent(s) and/or therapeutic agent(s) accumulate in cells expressing CD206. By this mechanism, a pharmaceutical formulation of the disclosure may be used to provide specific delivery of a diagnostic, imaging agent, or therapy to the infection, lung disease, fibrosis, inflammation, arthritis, or cancer.

The pharmaceutical formulation, B12 and IF are as described in Section I above. The subject, the cancer, the respiratory infection and the administration of the pharmaceutical formulation are described below.

(a) subject

A pharmaceutical formulation of the disclosure may be administered to a subject that is a human, a livestock animal, a companion animal, a lab animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In preferred embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc.

(b) cancer

A pharmaceutical formulation of the disclosure may be used to treat or recognize a tumor derived from a neoplasm or a cancer. In a specific embodiment, the tumor expresses CD206. For example, in embodiments where IF is conjugated to imaging agent(s) and/or therapeutic agent(s), the IF conjugate administered binds CD206. Alternatively, in embodiments where B12 or B12 analog is conjugated to imaging agent(s) and/or therapeutic agent(s), an IF complex comprising the B12/B12 analog conjugate binds CD206. As detailed in Section I, the IF and B12/B12 analog may be pre-bound to form complex (i.e., the complex is part of the pharmaceutical formulation) or formed in vivo following administration. The IF conjugate or IF complex is then internalized and the imaging agent(s) and/or therapeutic agent(s) is accumulated in cells expressing CD206. By this mechanism, a pharmaceutical formulation of the disclosure may be used to treat or recognize a tumor. CD206 has been shown to be expressed on liver cells and macrophages. However, any other neoplasm that expresses CD206 may also be used in the methods of the invention.

“Neoplasm” is any tissue, or cell thereof, characterized by abnormal growth as a result of excessive cell division. The neoplasm may be malignant or benign, the cancer may be primary or metastatic; the neoplasm or cancer may be early stage or late stage. Non-limiting examples of neoplasms or cancers that may be treated or detected, provided they express cubilin, include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenstrom), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), enknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood). In a preferred embodiment, the cancer is selected from the group consisting of bladder carcinoma, breast carcinoma, cervical carcinoma, cholangiocarcinoma, colorectal carcinoma, esophageal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head, a neck tumor, and a solid tumor.

### (c) Respiratory Infection

A pharmaceutical formulation of the disclosure may be used to treat, stabilize, prevent, diagnose or image a respiratory infection. The respiratory infection may be a bacterial, viral, fungal, or parasitic infection.

In some embodiments, the infection is a bacterial, viral, fungal, or parasitic infection of cells expressing CD206. CD206 has been shown to be expressed on alveolar macrophages and immature dendritic cells. However, any other respiratory cells that express CD206 may also be used in the methods of the invention. For example, in embodiments where IF is conjugated to imaging agent(s) and/or therapeutic agent(s), the IF conjugate administered binds CD206. Alternatively, in embodiments where B12 or B12 analog is conjugated to imaging agent(s) and/or therapeutic agent(s), an IF complex comprising the B12/B12 analog conjugate binds CD206. As detailed in Section I, the IF and B12/B12 analog may be pre-bound to form complex (i.e., the complex is part of the pharmaceutical formulation) or formed in vivo following administration. The IF conjugate or IF complex is then internalized and the imaging agent(s) and/or therapeutic agent(s) is accumulated in cells expressing CD206. By this mechanism, a pharmaceutical formulation of the disclosure may be used to treat, diagnose or image infected respiratory cells.

In certain embodiments, the infection is a viral infection. In particular embodiments, the viral infection is a coronavirus infection. In a specific embodiment, the viral infection is COVID-19, SARS, MERS, or any combination thereof.

Non-limiting examples of suitable therapeutic agents to treat a viral infection include antibiotics, anti-inflammatories, anti-viral agents, therapeutic antibodies, chemokines, cytokines, and the like. In certain embodiments, the viral infection is a coronovirus infection, in particular COVID-19, and the therapeutic agent is chloroquine, a chloroquine derivative, colchicine, corticosteroids, hydroxychloroquine, interferon (e.g., interferon beta, interferon I, interferon III, interferon alpha 2b), invermectin, lopinavir, oseltamivir, protease inhibitor (e.g., TMPRSS2, camostat mesylate, etc.), remdesivir, ribavirin, ritonavir, anti-IL-6 receptor antibodies (e.g., sarilumab), REGN-EB3, TLR4 antagonists, and the like. In a specific embodiment, the viral infection is a coronovirus infection, in particular COVID-19, and the therapeutic agent is chloroquine, a chloroquine derivative, or hydroxychloroquine.

### (d) Administration

In certain aspects, a pharmacologically effective amount of a pharmaceutical formulation of the disclosure may be administered to a subject. In some embodiments, a pharmacologically effective amount of a pharmaceutical formulation of the disclosure may be parenterally administered to a subject. Parenteral administration is performed using standard effective techniques. Parenteral administration includes but is not limited to subcutaneous, intravenous, intramuscular, intradermal, intra-arterial, intraosseous, intraperitoneal, or intrathecal injection, or infusion techniques. Effective parenteral systemic delivery by intravenous injection is a preferred method of administration to a subject. Suitable vehicles for such injections are straightforward. In some embodiments, a pharmacologically effective amount of a pharmaceutical formulation of the disclosure may be administered to a subject by inhalation. Inhalation is performed using standard effective techniques, including but not limited to vaping, a nasal spray, metered-dose inhaler, a dry-powder inhaler, a nebulizer, an atomizer, and the like.

Pharmaceutical formulations for effective administration are deliberately designed to be appropriate for the selected mode of administration, and pharmaceutically acceptable excipients such as compatible carriers, dispersing agents, buffers, surfactants, propellants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. It may be particularly useful to alter the solubility characteristics of the composition useful in this discovery, making it more lipophilic, for example, by encapsulating it in liposomes or by blocking polar groups.

For therapeutic applications, a therapeutically effective amount of a pharmaceutical formulation of the disclosure is administered to a subject. A “therapeutically effective amount” may be an amount of the therapeutic composition sufficient to produce a measurable biological response (e.g., a microbial response, an immunomodulary response, an anti-angiogenic response, a cytotoxic response, or tumor regression). Alternatively, a “therapeutically effective amount” may be an amount of the therapeutic composition sufficient to produce a measurable decrease in CD206 function. Actual dosage levels of active ingredients in a therapeutic composition of the disclosure can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, tumor size and longevity, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

For diagnostic applications, a detectable amount of a pharmaceutical formulation of the disclosure is administered to a subject. A “detectable amount”, as used herein to refer to a diagnostic composition, refers to a dose of such a pharmaceutical formulation that the presence of the pharmaceutical formulation can be determined in vivo or in vitro. A detectable amount will vary according to a variety of factors, including but not limited to chemical features of the drug being labeled, the imaging agent, labeling methods, the method of imaging and parameters related thereto, metabolism of the labeled drug in the subject, the stability of the label (e.g. the half-life of a radionuclide label), the time elapsed following administration of the drug and/or labeled peptide prior to imaging, the route of drug administration, the physical condition and prior medical history of the subject, and the size and longevity of the tumor or suspected tumor. Thus, a detectable amount can vary and can be tailored to a particular application. After study of the present disclosure, it is within the skill of one in the art to determine such a detectable amount.

A pharmaceutical formulation comprising IF may be administered at a concentration from about 0.1 pM to about 500 pM. For example, a composition comprising IF may be administered at a concentration of about 0.1 pM, about 0.2 pM, about 0.3 pM, about 0.4 pM, about 0.5 pM, about 0.6 pM, about 0.7 pM, about 0.8 pM, about 0.9 pM, about 1 pM, about 1.5 pM, about 2 pM, about 2.5 pM, about 3 pM, about 3.5 pM, about 4 pM, about 4.5 pM, about 5 pM, about 5.5 pM, about 6 pM, about 6.5 pM, about 7 pM, about 7.5 pM, about 8 pM, about 8.5 pM, about 9 pM, about 9.5 pM or about 10 pM. Alternatively, a composition comprising IF may be administered at a concentration of about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 150 pM, about 200 pM, about 250 pM, about 300 pM, about 350 pM, about 400 pM, about 450 pM, or about 500 pM. In a specific embodiment, a composition comprising IF may be administered at a concentration of about 1 pM. In another specific embodiment, a composition comprising IF may be administered at a concentration of about 4 pM. In still another specific embodiment, a composition comprising IF may be administered at a concentration from about 1 pM to about 10 pM. In still yet another specific embodiment, a composition comprising IF may be administered at a concentration from about 10 pM to about 50 pM. In other embodiments, a composition comprising IF may be administered at a concentration from about 50 pM to about 500 pM.

Typical dosage levels can and will vary and may be determined and optimized using standard clinical techniques and will be dependent in part on the imaging agent and/or therapeutic agent utilized and on the mode of administration.

The frequency of dosing may be daily or once, twice, three times or more per day, per week or per month, as needed as to effectively treat the symptoms. The timing of administration of the treatment relative to the disease itself and duration of treatment will be determined by the circumstances surrounding the case. Treatment could begin immediately, such as at the site of the injury as administered by emergency medical personnel. Treatment could begin in a hospital or clinic itself, or at a later time after discharge from the hospital or after being seen in an outpatient clinic. Duration of treatment could range from a single dose administered on a one-time basis to a life-long course of therapeutic treatments.

Although the foregoing methods appear the most convenient and most appropriate and effective for administration of the composition, by suitable adaptation, other effective techniques for administration may be employed provided proper formulation is utilized herein.

In addition, it may be desirable to employ controlled release formulations using biodegradable films and matrices, or osmotic mini-pumps, or delivery systems based on dextran beads, alginate, or collagen.

### III. Statements

Statement 1: A pharmaceutical formulation comprising (a) recombinantly produced intrinsic factor (IF) with a glycosylation pattern that enables binding to CD206, optional B12 or B12 analog, and at least one therapeutic, diagnostic, or imaging agent; wherein the IF is conjugated to a therapeutic, diagnostic, or imaging agent; or (b) recombinantly produced intrinsic factor (IF) with a glycosylation pattern that enables binding to CD206, B12 or B12 analog, and at least one therapeutic, diagnostic, or imaging agent; wherein the B12 or B12 analog is conjugated to a therapeutic, diagnostic, or imaging agent.

Statement 2: The pharmaceutical formulation of statement 1, wherein the IF is complexed to the B12 or B12 analog.

Statement 3: The pharmaceutical formulation of statement 1, wherein the IF is recombinantly produced in a plant.

Statement 4: The pharmaceutical formulation of statement 3, wherein the plant is Arabidopsis thaliana or Nicotiana benthamiana.

Statement 5: The pharmaceutical formulation of any one of the previous statements, wherein the IF is glycosylated with a(1-3)-fucose, xylose, mannose and n-acetylglucosamine.

Statement 6: The pharmaceutical formulation of statement 5, wherein the IF is glycosylated with a(1-3)-fucose, xylose, mannose and n-acetylglucosamine in ratios of about 0.17: about 0.18: about 1.0: about 0.24, respectively.

Statement 7: The pharmaceutical formulation of any one of the preceding statements, wherein the binding of IF to CD206 is not affected by endogenous B12 levels.

Statement 8: The pharmaceutical formulation of any one of the preceding statements, wherein the imaging agent is a radionuclide.

Statement 9: The pharmaceutical formulation of statement 8, wherein the radionuclide is selected from the group consisting of copper-64, zirconium-89, yttrium-86, yttrium-90, technetium-99m, iodine-125, iodine-131, lutetium-177, rhenium-186 and rhenium-188.

Statement 10: The pharmaceutical formulation of statement 8, wherein the radionuclide is also a therapeutic agent.

Statement 11: The pharmaceutical formulation of any one of statements 1 to 7, wherein the pharmaceutical formulation comprises a therapeutic agent selected from an anti-inflammatory agent, an anti-viral agent, an antibiotic.

Statement 12: The pharmaceutical formulation of statement 11, wherein the therapeutic agent is a nucleic acid, a small molecule, an antibody, or a polypeptide.

Statement 13: The pharmaceutical formulation of any one of the preceding statements, further comprising one or more pharmaceutically acceptable diluents, excipients, and/or carriers.

Statement 14: The pharmaceutical formulation of any one of the preceding statements, further comprising a chelator.

Statement 15: A method of treating microbial infection, lung disease, inflammation, fibrosis, arthritis or cancer in a subject, the method comprising administering to the subject a pharmaceutical formulation of any of statements 1-14, wherein the IF binds to CD206 in the liver or on macrophages, or skin epithelia of the subject.

Statement 16: The method of statement 15, wherein the therapeutic agent is selected from an anti-inflammatory agent, an anti-viral agent, an antibiotic.

Statement 17: The method of statement 15 or 16, wherein the therapeutic agent is a nucleic acid, a small molecule, an antibody, or a polypeptide.

Statement 18: The method of statement 15, wherein the therapeutic agent is a radionuclide.

Statement 19: A method of delivering a therapeutic, diagnostic, or imaging agent to a cell that expresses CD206 in a subject, the method comprising administering a pharmaceutical formulation of any of statements 1-14 to the subject.

Statement 20: The method of statement 19, wherein the cell is a liver cell or a macrophage.

Statement 21: The method of statement 20, wherein the cell is an alveolar macrophage.

Statement 22: The method of any one of statements 19 to 21, wherein the therapeutic agent is selected from an anti-inflammatory agent, an anti-viral agent, an antibiotic.

Statement 23: The method of any one of statements 19 to 22, wherein the therapeutic agent is a nucleic acid, a small molecule, an antibody, or a polypeptide.

Statement 24: A method of modulating CD206 function, the method comprising administering a pharmaceutical formulation of any of statements 1-14 to a subject.

Statement 25: A method of detecting microbial infection, lung disease, inflammation, arthritis, fibrosis or cancer in a subject, the method comprising: (a) administering to the subject a pharmaceutical formulation of any of statements 1-14, wherein the pharmaceutical formulation comprises an imaging agent; and (b) detecting the imaging agent, wherein the presence of the imaging agent indicates the presence of microbial infection, arthritis or cancer in the subject.

Statement 26: The method of statement 25, wherein the imaging agent is a radionuclide.

Statement 27: The method of statement 26, wherein the detecting comprises detecting the radionuclide label using positron emission tomography, single photon emission computed tomography, gamma camera imaging, or rectilinear scanning.

Statement 28: A method of treating microbial infection, fibrosis, lung disease, inflammation, arthritis or cancer in a subject, the method comprising administering to the subject a pharmaceutical formulation of any of statements 1-14, wherein the pharmaceutical formulation comprises a therapeutic agent.

Statement 29: The method of statement 28, wherein the therapeutic agent is a radionuclide.

Statement 30: The method of statement 28, wherein the therapeutic agent is selected from an anti-inflammatory agent, an anti-viral agent, an antibiotic.

Statement 31: The method of statement 28 or 30, wherein the therapeutic agent is a nucleic acid, a small molecule, an antibody, or a polypeptide.

Statement 32: A method of delivering B12 to a cell that expresses CD206 in a subject, the method comprising administering a pharmaceutical formulation of any of statements 1-14 to the subject.

Statement 33: The method of statement 32, wherein the cell is a liver cell or a macrophage.

Statement 34: The method of statement 33, wherein the macrophage is an alveolar macrophage.

Statement 35: The method of any one of statements 32 to 34, wherein the B12 is conjugated to an imaging agent and/or therapeutic agent.

Statement 36: The method of any one of statements 15 to 35, wherein the administration is oral.

Statement 37: The method of any one of statements 15 to 35, wherein the administration is topical.

Statement 38: The method of any one of statements 15 to 35, wherein the administration is intravenous.

Statement 39: The method of any one of statements 15 to 35, wherein the administration is parenteral.

Statement 36: The method of any one of statements 15 to 35, wherein the administration is by inhalation.

Statement 37: A method of delivering a therapeutic, diagnostic, or imaging agent to a liver of a subject, the method comprising: administering to the subject a pharmaceutical formulation of any of statements 1-14, wherein the IF binds to CD206 in the liver of the subject.

Statement 38: A method of delivering a therapeutic, diagnostic, or imaging agent to a kidney or a kidney cell of a subject, the method comprising: administering to the subject a pharmaceutical formulation of any of statements 1-14.

Statement 39: A method of delivering a therapeutic, diagnostic, or imaging agent to a lung of a subject, the method comprising: administering to the subject a pharmaceutical formulation of any of statements 1-14.

Statement 40: The method of statement 37, 38 or 39, wherein the administration is intravenous.

Statement 41: The method of statement 37, 38, or 39, wherein the administration is oral.

Statement 42: The method of statement 37, 38, or 39, wherein the administration is parenteral.

Statement 43: The method of any one of statements 37 to 42, wherein the imaging agent is a radionuclide.

Statement 44: The method of any one of statements 37 to 43, wherein the imaging agent is detected using positron emission tomography, single photon emission computed tomography, gamma camera imaging, or rectilinear scanning.

Statement 45: The method of any one of statements 37 to 42, wherein the therapeutic agent is selected from an anti-inflammatory agent, an anti-viral agent, an antibiotic.

Statement 46: The method of any one of statements 37 to 42 or 45, wherein the therapeutic agent is a nucleic acid, a small molecule, an antibody, or a polypeptide.

Statement 47: A method of delivering a therapeutic, diagnostic, or imaging agent to a lung of a subject, the method comprising: administering to the subject a pharmaceutical formulation of any of statements 1-14, wherein the IF binds to CD206 in the lung of the subject.

Statement 48: The method of statement 47, wherein the administration is by inhalation.

Statement 49: The method of statement 47 or 48, wherein the IF binds to alveolar macrophages expressing CD206 in the lung of the subject.

Statement 50: The method of any one of statements 47 to 49, wherein the imaging agent is a radionuclide.

Statement 51: The method of any one of statements 47 to 50, wherein the imaging agent is detected using positron emission tomography, single photon emission computed tomography, gamma camera imaging, or rectilinear scanning.

Statement 52: The method of any one of statements 47 to 49, wherein the therapeutic agent is selected from an anti-inflammatory agent, an anti-viral agent, an antibiotic.

Statement 53: The method of any one of statements 47 to 49 or 52, wherein the therapeutic agent is a nucleic acid, a small molecule, an antibody, or a polypeptide.

Statement 54: A method of treating a respiratory infection in a subject, the method comprising administering by inhalation to the subject a pharmaceutical formulation of any of statements 1-14, wherein the pharmaceutical formulation comprises a therapeutic agent.

Statement 55: The method of statement 54, wherein the respiratory infection is a viral infection.

Statement 56: The method of statement 55, wherein the viral infection is a coronovirus infection.

Statement 57: The method of statement 56, wherein the coronovirus infection is SARS, MERS or COVID-19.

Statement 58: The method of statement 56, wherein the coronovirus infection is COVID-19.

Statement 59: The method of any one of statements 54 to 58, wherein the therapeutic agent is conjugated to B12.

Statement 60: The method of statement 59, wherein the therapeutic agent is conjugated to B12 directly or indirectly on the A ring at the b-position, on the C ring at the e-position, on the ribose unit at the 5′-hydroxyl group, or on the cobalt cation

Statement 61: The method of statement 59, wherein the therapeutic agent is conjugated to B12 directly or indirectly on the ribose unit at the 5′-hydroxyl group.

Statement 62: The method of any one of statements 54 to 61, wherein the therapeutic agent is selected from an anti-inflammatory agent, an anti-viral agent, an antibiotic.

Statement 63: The method of any one of statements 54 to 62, wherein the therapeutic agent is a nucleic acid, a small molecule, an antibody, or a polypeptide.

Statement 64: The method of statement 62, wherein the therapeutic agent is chloroquine, hydroxychloroquine or a derivative thereof.

Statement 65: The method of any one of statements 1 to 53, wherein the therapeutic agent is conjugated to B12.

Statement 66: The method of statement 65, wherein the therapeutic agent is conjugated to B12 directly or indirectly on the A ring at the b-position, on the C ring at the e-position, on the ribose unit at the 5′-hydroxyl group, or on the cobalt cation

Statement 67: The method of statement 65, wherein the therapeutic agent is conjugated to B12 directly or indirectly on the ribose unit at the 5′-hydroxyl group.

Statement 68: The method of any one of statements 1 to 53, wherein the therapeutic agent is conjugated to IF directly or indirectly.

### IV. Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The term “alkyl” as used herein describes groups which are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkenyl” as used herein describes groups which are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The term “alkoxide” or “alkoxy” as used herein is the conjugate base of an alcohol. The alcohol may be straight chain, branched, cyclic, and includes aryloxy compounds.

The term “alkynyl” as used herein describes groups which are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aromatic” as used herein alone or as part of another group denotes optionally substituted homo- or heterocyclic conjugated planar ring or ring system comprising delocalized electrons. These aromatic groups are preferably monocyclic (e.g., furan or benzene), bicyclic, or tricyclic groups containing from 5 to 14 atoms in the ring portion. The term “aromatic” encompasses “aryl” groups defined below.

The terms “aryl” or “Ar” as used herein alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The terms “carbocyclo” or “carbocyclic” as used herein alone or as part of another group denote optionally substituted, aromatic or non-aromatic, homocyclic ring or ring system in which all of the atoms in the ring are carbon, with preferably 5 or 6 carbon atoms in each ring. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halo, heterocyclo, hydroxyl, keto, ketal, phospho, nitro, and thio.

The terms “halogen” or “halo” as used herein alone or as part of another group refer to chlorine, bromine, fluorine, and iodine.

The term “heteroatom” refers to atoms other than carbon and hydrogen.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms and/or 1 to 4 nitrogen atoms in the ring, and is bonded to the remainder of the molecule through a carbon. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halo, heterocyclo, hydroxyl, keto, ketal, phospho, nitro, and thio.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halo, heterocyclo, hydroxyl, keto, ketal, phospho, nitro, and thio.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted, or replaced, with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halo, heterocyclo, hydroxyl, keto, ketal, phospho, nitro, and thio.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction to the Example

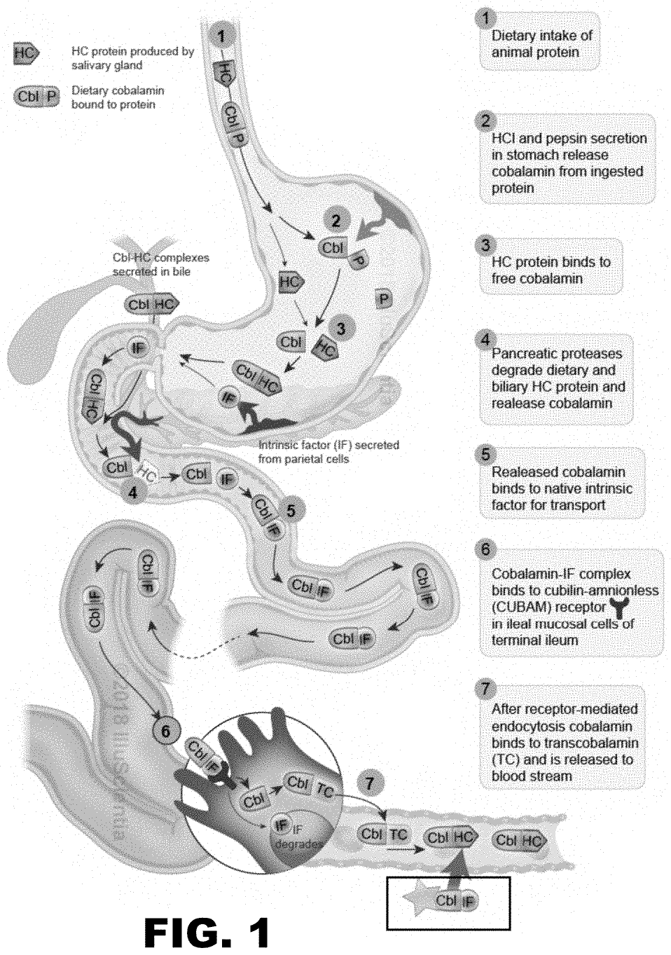

A basic understanding of the dietary pathway of vitamin B12 (B12) is in place (FIG. 1).[20] Mammals have developed a complex dietary uptake pathway for B12 involving a series of transport proteins and specific receptors across various tissues and organs. Transport and delivery of B12 utilizes three primary carrier proteins: haptocorrin (HC; K_{d}=0.01 pM), intrinsic factor (IF; K_{d}=1 pM), and transcobalamin (TC; K_{d}=0.005 pM), each responsible for carrying a single B12 molecule.[20] B12 is initially released from food by the action of peptic enzymes and the acidic environment of the gastrointestinal system and then bound by HC (Holo-HC). Holo-HC travels from the stomach to the duodenum, where pancreatic digestion effects B12 release, whereupon it is bound by gastric intrinsic factor (IF). IF is a ˜50 kDa glycosylated protein that is secreted from parietal cells of the gastric mucosa and is resistant to pancreatic enzymes.[16, 20]

Once B12 is bound to IF, it facilitates intestinal transport and passage across the ileal enterocyte. This passage occurs via receptor-mediated endocytosis through the IF-B12 receptor cubilin (CUBN) combined with a transmembrane protein amnionless.[21,22] Following internalization, IF is degraded by lysosomal proteases and B12 is released into the blood stream, either as free B12 or pre-bound to TC.[20,23] Cells that require B12 express the holo-TC receptor, CD320. Upon internalization, TC is degraded and B12 is transported from the lysosome for cellular use.

Herein, the effects of systemic administration of B12 conjugates pre-bound to recombinant human gastric IF were investigated (FIG. 1). A number of possible outcomes to pre-binding B12 to gastric IF and injecting it systemically were hypothesized. The first outcome postulated was that IF pre-binding would prevent blood TC binding and hence would not affect, or be affected by, endogenous B12 levels, a concern in the field given the possibility that long-term use of a B12-conjugate that might be bound to TC would result in reduced capacity to deliver dietary B12 to proliferating cells, as necessary.[24] Such administration would also likely target the only known holo-IF receptor, CUBN, located in the ileum in the enterocyte as described for dietary uptake, but also in the proximal tubules (PT) of the kidney, where it plays a role in reabsorption of such ligands as albumin, transferrin, vitamin D binding protein, apolipoprotein AI, amongst others.[25] Expression of CUBN elsewhere is limited, including the human inner ear[26] and yolk sac.[27]

This systemic approach focused on whether plant IF could be used to target (1) renal cell carcinoma (RCC), including metastasized RCC, of which ˜80% stems from kidney PT or (2) receptors tied to its specific plant glycosylation profile such as the asialoglycoprotein receptor (ASGPR)[28] or CD206 receptor (MR; MRC1).[29]

Before beginning such work, it was necessary to ensure access to IF that (1) was available commercially on a large-scale (i.e. 30-50 mg quantities) necessary to conduct, and ultimately translate, the work, and (2) that it was in the apo- (i.e. no pre-bound B12) form, to allow binding of the desired B12-conjugates, which in this case are radio-probes of ⁸⁹Zirconium-B12 (⁸⁹Zr-B12), vide infra.[30] To achieve this, the only available source meeting our criteria was human recombinant IF (hrIF) produced in the plant Arabidopsis thaliana.[31] Expression in plants produces apo-IF, given plants are a rare organism that does not use B12, minimizing holo-IF production in situ. Questions to be explored with A. thaliana produced hrIF included the glycosylation profile of such a protein and the effects of such glycosylation on receptor targeting in vivo, as noted above, and whether this profile negated, complemented or refocused the CUBN targeting hypothesis noted above.

The full glycosylation content of hrIF produced in A. thaliana is established herein and it is demonstrated that such glycosylation facilitates targeting of the liver in mice, likely through the CD206 receptor and to a lesser extent the kidney, as would be predicted via CUBN uptake. CD206 is a member of the C-type lectin superfamily and is produced by most tissues macrophages and select endothelial and dendritic cells and plays a key role in the innate and adaptive immune response in humans.[32] Conversely, tumor-associated macrophages (TAM) positive for CD206 have been shown to contribute to tumor growth, metastasis, and relapse.[33] CD206 has also been shown to be involved in leukocyte trafficking and inflammation. Thus, CD206 has become an attractive target in precision imaging, diagnosis and/or therapy of diseases including microbial infection, arthritis, and cancer.[29,34]

CD206 is a pattern recognition receptor that can facilitate the endocytosis of target glycan antigens with terminal mannose, fucose or N-acetylglucosamine.[28] The post-translation glycan specific modification of proteins produced in plants such as A. thaliana commonly involves all three such sugars, making glycosylated proteins recombinantly expressed via such sources a potential source of CD206 specific targeting. In addition, it is demonstrated herein, via studies comparing radiolabeled B12 conjugate to such a conjugate pre-bound to IF in mice on replete or deplete B12 diets, that the hrIF pre-bound conjugate is not affected by endogenous B12 levels, unlike the free B12-conjugate. The question of whether utilizing B12-conjugates as pharmaceuticals would interfere with functional B12 levels, especially with prolonged use, has been a significant one in the field. Demonstrating that B12 conjugates pre-bound to IF could be used without such interference would be a first and overcome a potentially limiting concern to their use.

### Example 1

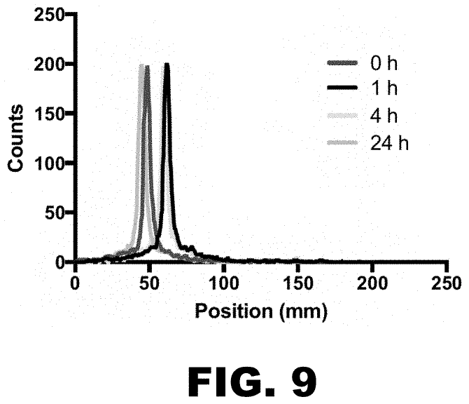

B12-DFO and B12-DFO-⁸⁹Zr (⁸⁹Zr-B12) were synthesized and characterized as previously reported with a final yield of 20 and 100%, respectively.[30] The specific activity of the tracer for studies herein was determined by titrating ⁸⁹Zr⁴⁺ and B12-DFO at different mole ratios with an achieved optimum specific activity of 250±20 mCi/μmol. Stability of the tracer was analyzed by incubating the IF-⁸⁹Zr-B12 in saline at physiological temperature and analyzing fractions up to 24 h using iTLC (FIG. 9). Results indicated that the IF-⁸⁹Zr-B12 tracer was stable to demetallation up to 24 h.

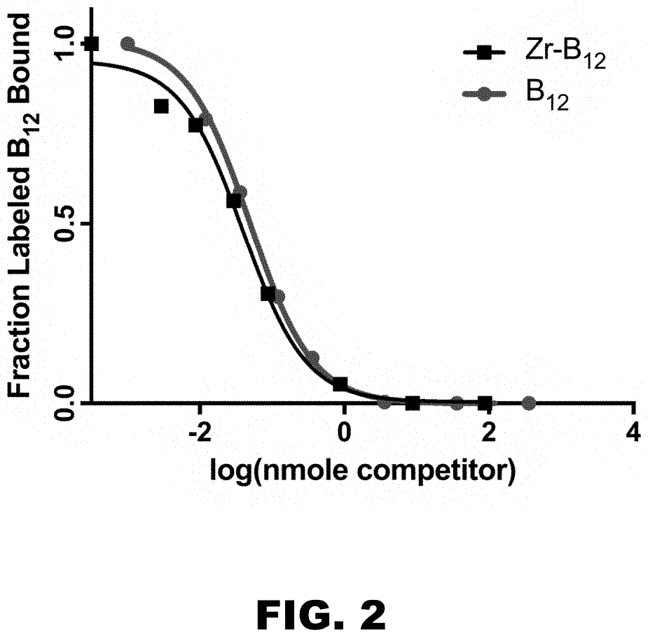

To confirm IF binding of ⁸⁹Zr-B12, a radiometric chase assay[15] was completed with a ‘cold’ ⁹¹Zr bound to B12 tracer (⁹¹Zr-B12) and compared to free B12, as cyanocobalamin (CN-B12) (FIG. 2). ⁹¹Zr-B12 was made using B12-DFO and chelated to ⁹¹ZrCl₄ at pH 7-7.5. IF binding of ⁹¹Zr-B12 was maintained at low nanomolar levels (1.57 nM), similar to CN-B12 control (1.36 nM).

The glycosylation of IF was examined by GC-MS (Table 1A and Table 1B). The sugars identified were α(1-3)-fucose, xylose, mannose and n-acetylglucosamine in the ratios 0.17:0.18:1.0:0.24, respectively.

TABLE 1A
GC-MS analysis of hrIF expressed in A. thaliana

| | Peak Area [nmol] | | Ratio |
|---|---|---|---|
| Sugar | Ave. | Detected | (Man = 1.0) |
| Fucose | 8471 | 11.2 | 0.17 |
| Xylose | 6156 | 11.8 | 0.18 |
| Mannose | 81654 | 66.7 | 1.0 |
| n-acetylglucosamine | 4508 | 16.0 | 0.24 |

TABLE 1B

| | Peak Area | Area ratio | | |
|---|---|---|---|---|
| | Average | Mono/IS | nmoles detected | Man = 1.0¹⁾ |
| Fuc | 8471 | 0.473 | 11.2 | 0.17 |
| Xyl | 6156 | 0.344 | 11.8 | 0.18 |
| Man | 81654 | 4.558 | 66.7 | 1.0 |
| Gal | n.d. | — | — | — |
| Glc | n.d. | — | — | — |
| GalNAc | n.d. | — | — | — |
| GlcNAc | 4508 | 0.252 | 16.0 | 0.24 |
| NeuAc | n.d. | — | — | — |
| Ara-IS | 17914 | — | — | — |

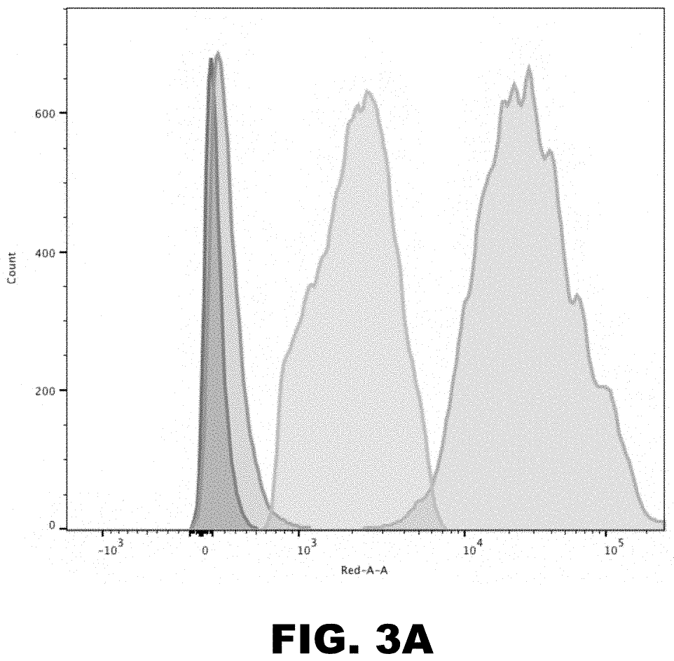

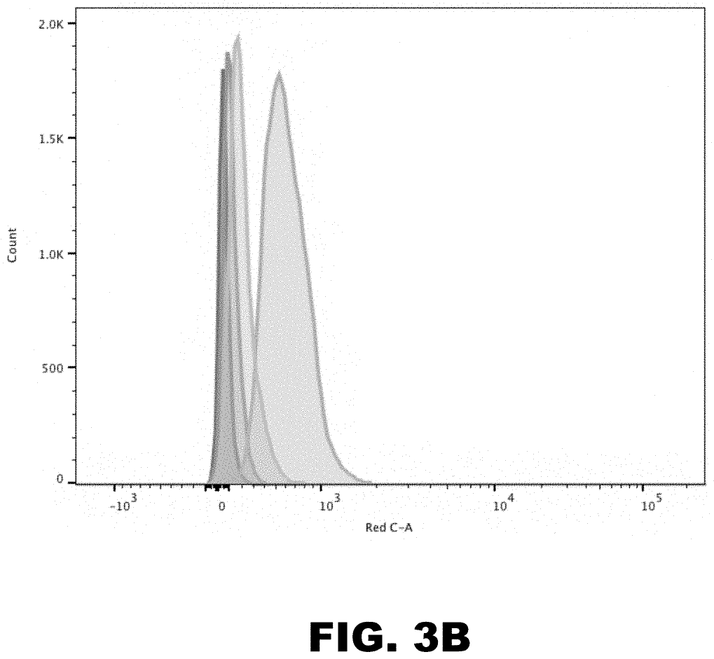

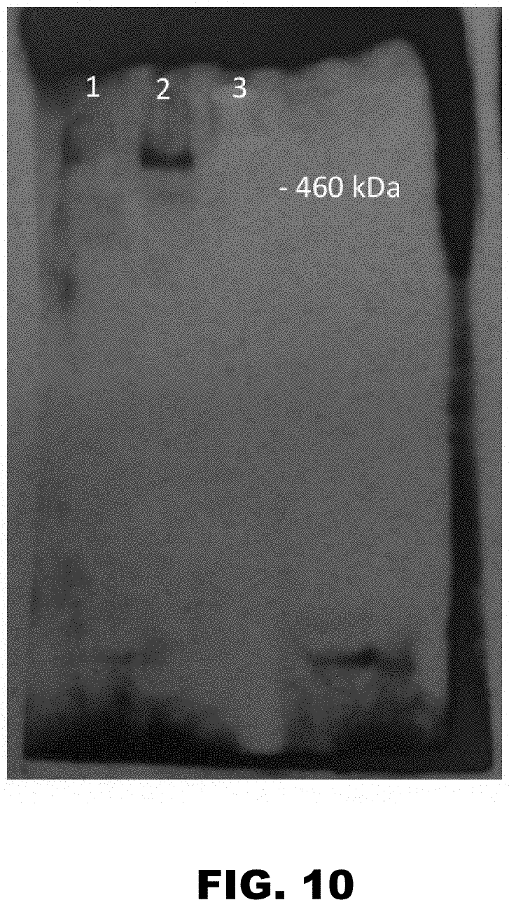

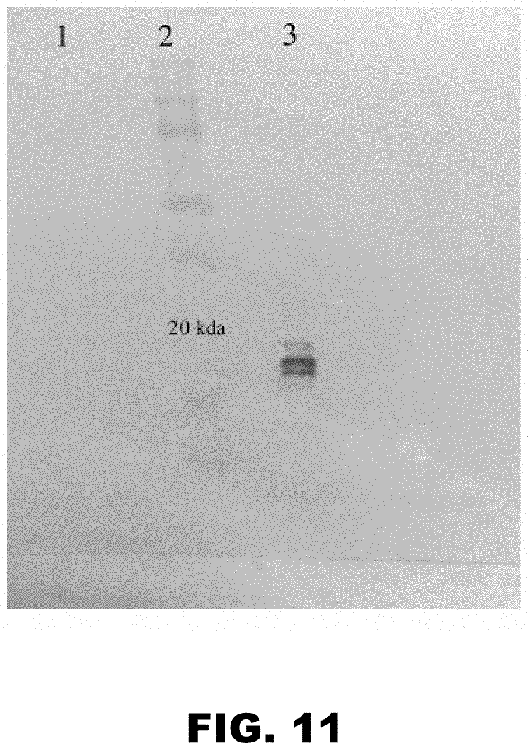

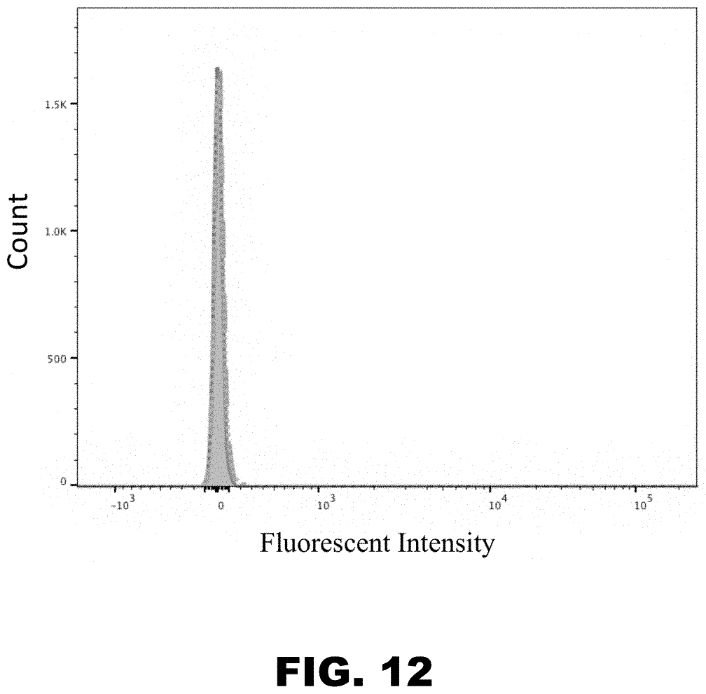

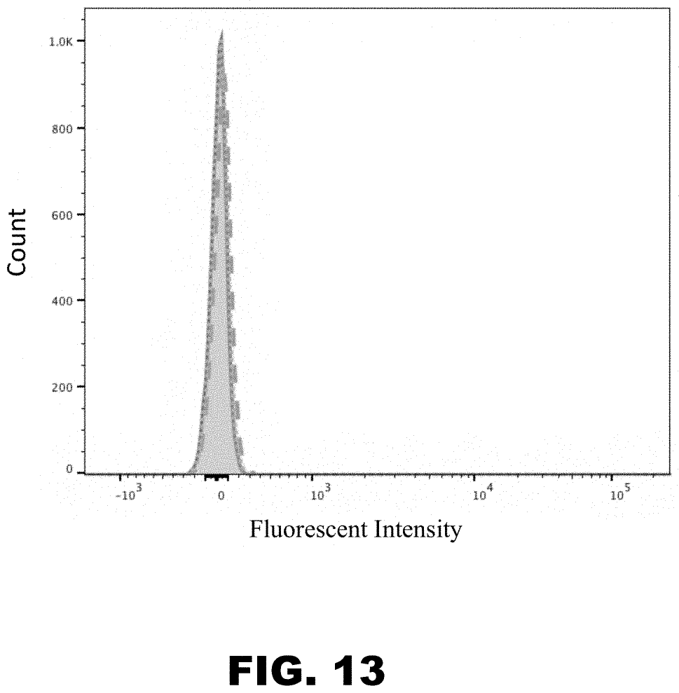

Cellular association via the typical holo-IF target receptor, CUBN, was conducted in CUBN positive, CD206 negative (see western blot, FIG. 10) BN16 (Brown Norway rat yolk) cells via flow cytometry using fluorescent B12-Cy5 to show functionalization of the IF-B12 complex in vitro (FIG. 3). Results showed no association of B12-Cy5 alone, and significant association of IF-B12-Cy5 at 37° C. Reduction in binding (or internalization) of IF-B12-Cy5 at 4° C. supported a receptor mediated internalization. No association/binding was observed in Chinese hamster ovary (CHO) cells (CUBN and CD206 free cells; FIG. 12) or in ASGPR positive (FIG. 11) HepG2 cells (FIG. 13).

Then, uptake in J774.A1 macrophage cells (CUBN− and CD206+) was investigated,[35] which again showed no binding of B12-Cy5 alone, and binding of IF-B12-Cy5 at 37° C. Adding mannan (2 mg/mL), 45 minutes prior to, and concomitant with IF-B12-Cy5 incubation, reduced IF-B12-Cy5 uptake (FIG. 3) supporting a mannose receptor mediated process.

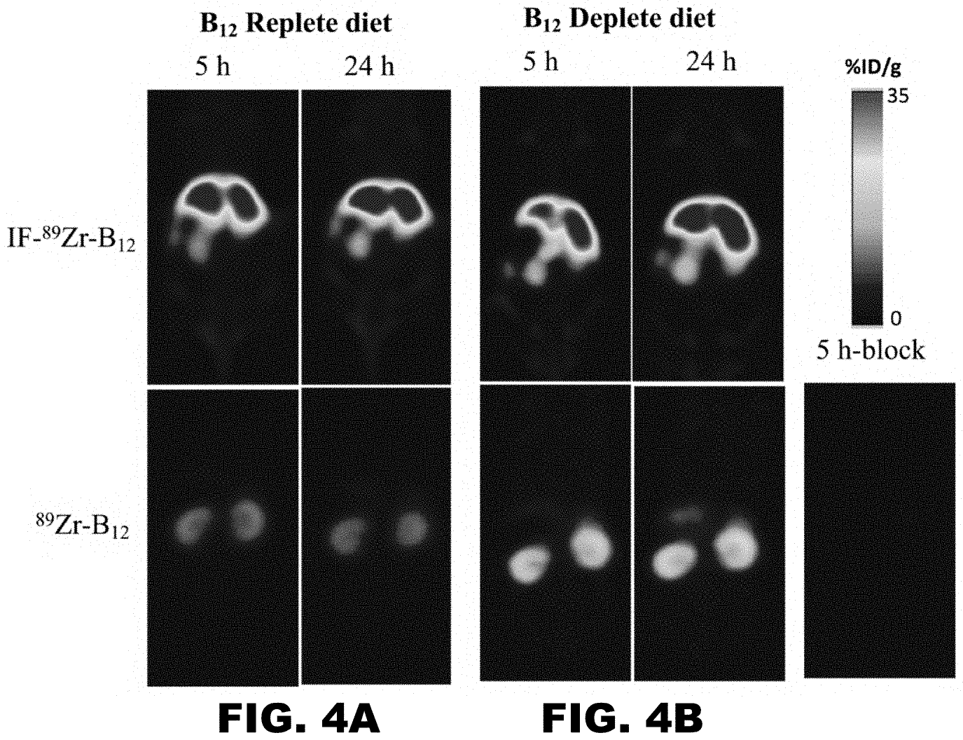

Upon completion of the synthesis and characterization of the ⁸⁹Zr conjugate of interest PET imaging studies were conducted. Initially, PET imaging was completed in nude athymic female mice on replete chow containing B12 at 1, 5 and 24 h p.i. (200-250 μCi/mouse via the tail vein) of IF-⁸⁹Zr-B12. As shown in FIG. 4 and Table 2 there was significant liver uptake at 5 h, which did not change over the subsequent 24 h. Experiments were duplicated in mice on a B12 deplete diet for 21 days. For IF-⁸⁹Zr-B12 the highest uptake was seen in the liver and kidneys and did not look significantly different than mice on replete diets (FIG. 4). However, in comparison to ⁸⁹Zr-B12 a change was observed with reduced kidney uptake noted in deplete animals (FIG. 4; Table 2).

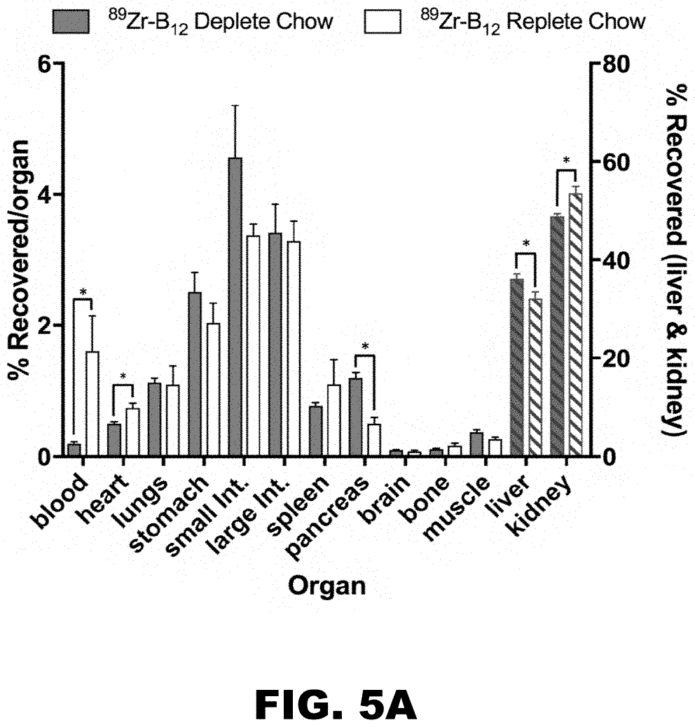

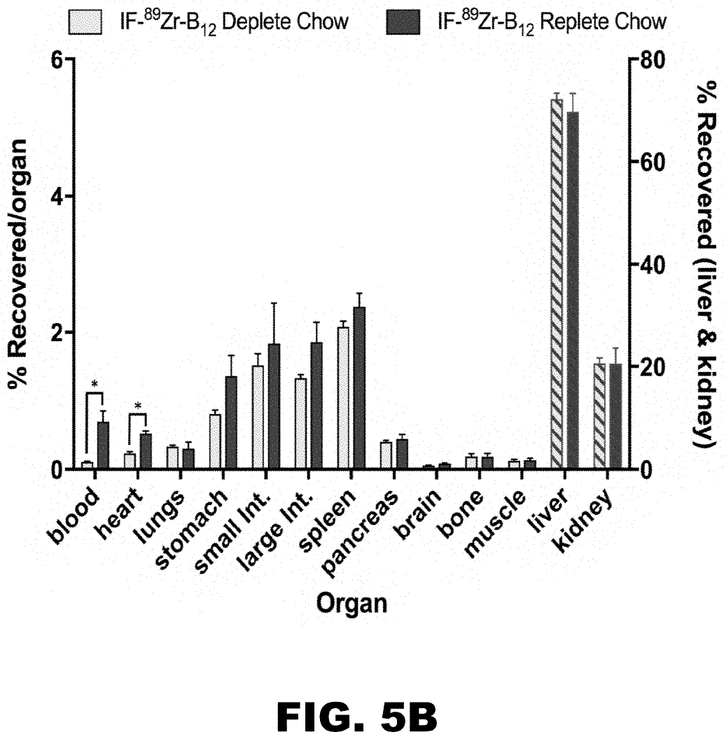

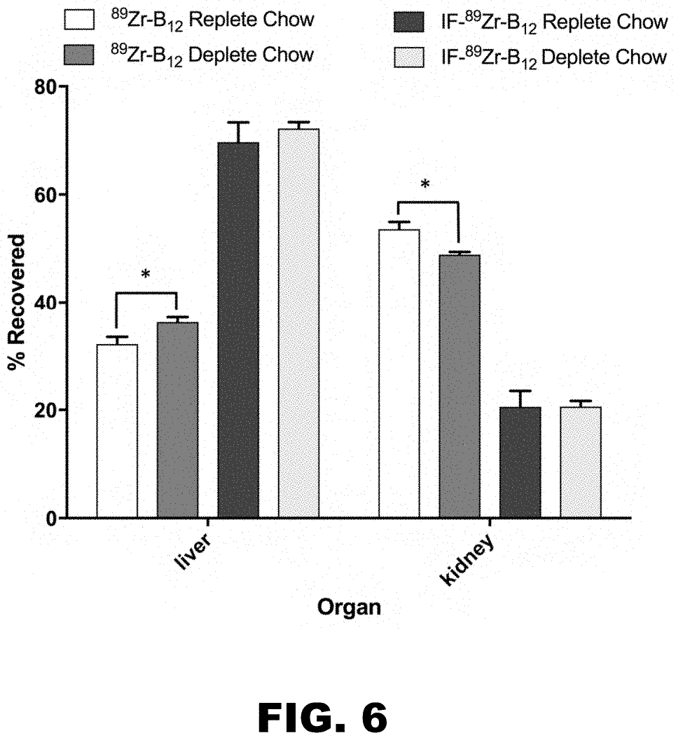

Due to the interesting uptake seen in PET imaging using IF-⁸⁹Zr-B12 in mice, ex vivo distribution was examined (FIG. 5 and FIG. 6 and Table 2). ⁸⁹Zr-B12 replete and deplete showed significant change in uptake within the liver, kidneys, blood, pancreas, and heart between the two mice models (B12 replete and deplete diets) (liver: 32.18±2.6 vs 36.24±1.8, kidney: 53.58±2.7 vs 48.89±1.0, blood: 1.60±1.0 vs 0.192±0.05, pancreas: 0.489±0.18 vs 1.19±0.15, heart: 0.740±0.14 vs 0.501±0.05, % recovered/organ for replete vs deplete; p≤0.05, n=4) (Table 2).

The IF-⁸⁹Zr-B12 injected mouse models showed significant change in uptake within the blood, and heart (blood: 0.69±0.31 vs 0.106±0.01, heart: 0.51±0.09 vs 0.23±0.04% recovered/organ for replete vs deplete; p≤0.05, n≥3). IF-⁸⁹Zr-B12 uptake in the liver, kidneys, spleen, and pancreas were not significantly different between the two models (liver uptake: 69.67±7.3 vs 72.22±2.0, kidneys: 20.56±5.9 vs 20.61±1.8, spleen: 2.37±0.40 vs 2.07±0.14, and pancreas: 0.43±0.12 vs 0.399±0.03% recovered/organ for replete vs deplete) (Table 2).

TABLE 2
Ex vivo tissue distribution of IF-⁸⁹Zr-B12 and ⁸⁹Zr-B12 in mice on a B12
deplete or replete diet at 24 h plotted as % recovered/organ as mean ± SD.

| | ⁸⁹Zr-B12 | IF-⁸⁹Zr-B12 | ⁸⁹Zr-B12 | IF-89Zr-B12 |
|---|---|---|---|---|
| Organs | Replete | Replete | Deplete | Deplete |
| Blood | 1.60 ± 1.07^{a} | 0.72 ± 0.26^{b} | 0.19 ± 0.05^{a} | 0.106 ± 0.01^{b} |
| Heart | 0.74 ± 0.14^{a} | 0.51 ± 0.09^{b} | 0.50 ± 0.05^{a} | 0.23 ± 0.04^{b} |
| Lungs | 1.09 ± 0.57 | 0.29 ± 0.19 | 1.12 ± 0.12 | 0.32 ± 0.03 |
| Liver | 32.18 ± 2.61^{a} | 69.67 ± 7.34 | 36.24 ± 1.88^{a} | 72.22 ± 2.02 |
| Kidney | 53.58 ± 2.72^{a} | 20.56 ± 5.90 | 48.88 ± 1.01^{a} | 20.61 ± 1.81 |
| Stomach | 2.03 ± 0.61 | 1.36 ± 0.60 | 2.51 ± 0.59 | 0.80 ± 0.09 |
| Small Int. | 3.37 ± 0.35 | 1.82 ± 1.20 | 4.55 ± 1.59 | 1.51 ± 0.28 |
| Large Int. | 3.28 ± 0.61 | 1.85 ± 0.58 | 3.41 ± 0.87 | 1.33 ± 0.08 |
| Spleen | 1.09 ± 0.75 | 2.37 ± 0.40 | 0.77 ± 0.10 | 2.07 ± 0.14 |
| Pancreas | 0.49 ± 0.18^{a} | 0.43 ± 0.12 | 1.19 ± 0.15^{a} | 0.39 ± 0.03 |
| Brain | 0.08 ± 0.03 | 0.08 ± 0.02 | 0.09 ± 0.01 | 0.05 ± 0.01 |
| Bone | 0.16 ± 0.07 | 0.18 ± 0.10 | 0.11 ± 0.02 | 0.18 ± 0.06 |
| Muscle | 0.26 ± 0.06 | 0.13 ± 0.06 | 0.37 ± 0.07 | 0.12 ± 0.03 |

^{a}p ≤ 0.05 between ⁸⁹Zr-B12 replete and deplete mouse models;
^{b}p ≤ 0.05 between IF-⁸⁹Zr-B12 replete and deplete mouse models.

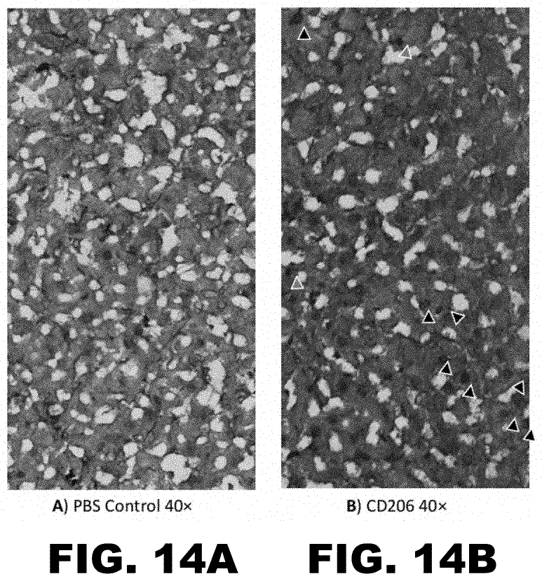

The presence of CD206 in the liver was further investigated through immunohistochemical (IHC) analyses. The anti-mannose receptor antibody exhibited positive staining for cell membrane and nuclear localization (FIG. 14) consistent with the presence of CD206 receptor.

### Discussion for Example 1

The apo-IF's glycosylation profile from A. thaliana was characterized given the postulated differences in glycol profile for human versus plant IF, and the role such sugars can play in terms of receptor recognition and binding, protein clearance, etc. GC-MS data showed a plant glycosylation profile of α(1-3)-fucose, xylose, mannose and n-acetylglucosamine in the ratios 0.17:0.18:1.0:0.24, respectively. Since galactose was not detected the most likely receptor causing the liver internalization of IF was the mannose receptor CD206, which recognizes fucose, mannose, and n-acetylglucosamine and is found in liver epithelial cells and macrophages. ASGPR is also highly expressed in the liver, however, this receptor recognizes terminal galactose, which was not present on the hrIF used herein.[33] Since this differs from a human glycosylation profile it was investigated if the glycosylation profile might alter IF's recognition in the body (should be only recognized by CUBN) and be recognized by the CD206.

To validate the proposed hypothesis, in vitro experiments with a fluorescent B12 conjugate, B12-Cy5, synthesized previously, were performed to allow the performance of quantitative flow cytometry experiments. It was confirmed that the IF-B12-Cy5 functioned as endogenous IF and that it was recognized by CUBN in the CUBN+ cell line BN16. Then, uptake in J774.A1 macrophage cells (CUBN− and CD206+), was investigated which indicated that IF-B12-Cy5 recognition is IF specific and supports the GC-MS sugar profile. In addition, a near complete block in uptake was observed when J774A.1 cells were incubated with an excess (2 mg/mL) of mannan, which is reported to reduce CD206 mediated uptake,[35] 45 minutes prior to incubation with IF-B12-Cy5, supporting that the uptake is mediated via the CD206 receptor (FIG. 3). CUBN and CD206 negative cell line CHO-K1 (confirmed by Western blot-data not shown) did not show any uptake (FIG. 12).

Since the in vitro studies confirmed the hypothesis, the investigation was continued in vivo using PET imaging. Upon completion of the synthesis, characterization, and stability studies ⁸⁹Zr-B12 and IF-⁸⁹Zr-B12 indicated that in vivo PET imaging studies could be conducted. Initially, PET imaging was completed in nude athymicfemale mice on replete chow containing B12 at 1, 5, and 24 h p.i. (200-250 μCi/mouse via the tail vein) of IF-⁸⁹Zr-B12 (data not shown). As shown in FIG. 4 and Table 2, there was significant liver uptake at 5 h, which did not change over the subsequent 24 h. Overall, the highest uptake was observed in the liver, compared to the control (⁸⁹Zr-B12 alone) which showed uptake primarily in the kidneys.

To more closely examine the effects of a B12 diet IF-⁸⁹Zr-B12 or ⁸⁹Zr-B12 were injected into nude athymic female mice on a B12 deplete diet for 21 days and PET imaging was completed on mice 24 h p.i. FIG. 4 shows PET imaging of IF-⁸⁹Zr-B12 and the control ⁸⁹Zr-B12. For IF-⁸⁹Zr-B12 the highest uptake was seen in the liver and kidneys and did not look significantly different than mice on replete diets. However, in comparison to ⁸⁹Zr-B12 a clear change was observed with higher uptake in the liver. To quantify this change biodistribution studies were conducted.

Due to the interesting uptake seen in PET imaging using IF-⁸⁹Zr-B12 and ⁸⁹Zr-B12 in mice ex vivo distribution was examined (FIG. 5 and FIG. 6 and Table 2). ⁸⁹Zr-B12 replete and deplete showed significant change in uptake within the liver, kidneys, blood, pancreas, and heart (p<0.05). The IF-⁸⁹Zr-B12 replete and deplete models showed significant change within the blood, and heart (p<0.05). To date most B12 experiments show high uptake in the kidneys with less uptake in the liver, the complex presented herein displays an altered pharmacokinetic (PK) and uptake profile for the IF-bound B12. This change in PK is most likely, in part, due to the CD206 receptor, highly expressed in the liver and macrophages (and confirmed FIG. 14), which recognize the specific glycosylation profile of A. thaliana produced recombinant human IF.

In conclusion, the absence of effect on IF uptake by endogenous B12 levels indicates that IF can allow for the use of B12 conjugate chemistry (i.e. B12 drug conjugates) while stepping out of the B12 ‘dietary’ pathway dependent on TC mediated cellular uptake. This use of IF would diminish the concern of developing B12 deficiency in subjects being dosed with B12 bioconjugates. The liver uptake seen in PET imaging and biodistribution when a radio-B12 complex of IF was administered was attributed to the terminal sugar being recognized by, most likely, the CD206 receptor, itself a major target for pharmaceutical intervention/targeting.

A new avenue of exploration to exploit the vitamin B12 pathway for pharmaceutical and/or probe development has successfully been developed.

### Methods for Example 1

Reagents: Reagents listed below were purchased and used without further manipulations: Dimethyl sulfoxide (DMSO, 99%, Sigma), Vitamin B12 (Cbl, ≥98%, Sigma), 1,10-carbonyl-di-(1,2,4-triazole) (CDT, ≥90%, Fluka), acetonitrile (MeCN, 99.8%, Pharmaco-Aaper), desferrioxamine mesylate (Sigma), F12-K media (VWR), Dulbecco's modified eagles medium (DMEM) (VWR), mannan (VWR).

Western blotting: Samples were run on a 12% acrylamide gel and then transferred to a nitrocellulose membrane using an iBlot (Invitrogen) dry blotting system. The membrane was blocked in a 5% nonfat powdered milk PBS-T solution (w/v) for one hour at room temperature prior to western blotting.

Antibodies: 1° Santa Cruz Biotechnology cubilin anti-goat polyclonal (1:200); Santa Cruz Biotechnology chicken anti-goat HRP conjugated (1:4000); anti-mannose (CD206) receptor antibody (abcam, ab64693); anti-asialoglycoprotein receptor (abcam, ab88042), HRP-conjugated goat anti-rabbit (abcam, ab6721).

RP-HPLC: RP-HPLC was performed using either an Agilent 1200 system or a Shimadzu Prominence with an Agilent Eclipse C₁₈ XBD analytical column (5 μm×4.6 mm×150 mm) using a 0-70% 0.1% aqueous TFA to MeCN gradient over 30 minutes.

Proton nuclear magnetic resonance: Proton nuclear magnetic resonance (¹H NMR) was performed using a 400 MHz Bruker spectrometer with the residual non-deuterated solvent peak as an internal standard.

MALDI-MS: Matrix assisted laser desorption ionization mass spectrometry (MALDI-MS) was conducted on a Bruker Autoflex III smartbeam using sinapinic acid (Sigma) as matrix. Quantification in solution used a Shimadzu BioSpec-Nano.

Flow cytometry: Flow cytometry analyses were carried out on a Becton Dickinson LSRII Cell Analyzer.

Cell culture: Cell lines J774A.1 (ATCC TIB-67; CD206 positive), CHO-K1 (ATCC CCL-61; control line) and HepG2 (SIGMA 85011430; ASGPR positive) were obtained from the American Type Culture Collection (ATCC). BN16 cells (cubilin positive) were kindly provided by Prof. Pierre Verroust (INSERM, Paris, France). J774A.1 and BN16 cells were cultured as adherent monolayers in DMEM supplemented with 10% FBS and 1% pen/strep (Penicillin-streptomycin solution with 10,000 units penicillin and 10 mg/mL streptomycin in 0.9% NaCl obtained from Thermo Fisher). CHO-K1 were cultured as adherent monolayers in F12-K supplemented with 10% FBS and 1% pen/strep. Cells were incubated at 37° C. with 5% C₀₂. Hank's balanced salt solution (HBSS) was purchased from Sigma. Charcoal stripped fetal bovine serum (FBS) and were purchased from Sigma.

Apo-hrIF: Xeragenx LLC (St. Louis, Mo., USA) supplied the apo-hrIF expressed in A. thaliana.

Analysis of radiotracer Analysis of the radiotracer was performed using C18 reverse phase high-pressure liquid chromatography (RP-HPLC, Agilent 1260 with manual injection) and instant thin layerchromatography (iTLC, Eckert & Ziegler Mini Scan). EDTA (50 mM) mobile phase was used for iTLC.

Mice: Female athymic nude mice (5-6 weeks old) were purchased from Envigo (Catalog #069). All animal experiments and manipulations were carried out according to the guidelines and regulations set by the Institutional Animal Use and Care Committee at Wayne State University, which is accredited by the Association for Assessment and Accreditation of Laboratory Animal Care (AAALAC). IACUC Protocol # for this work 17-07-302.

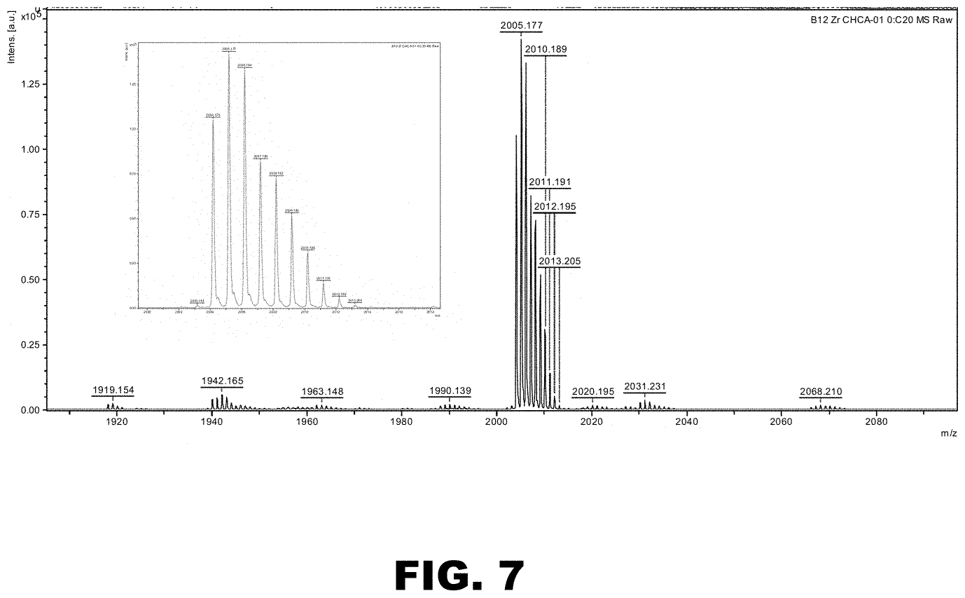

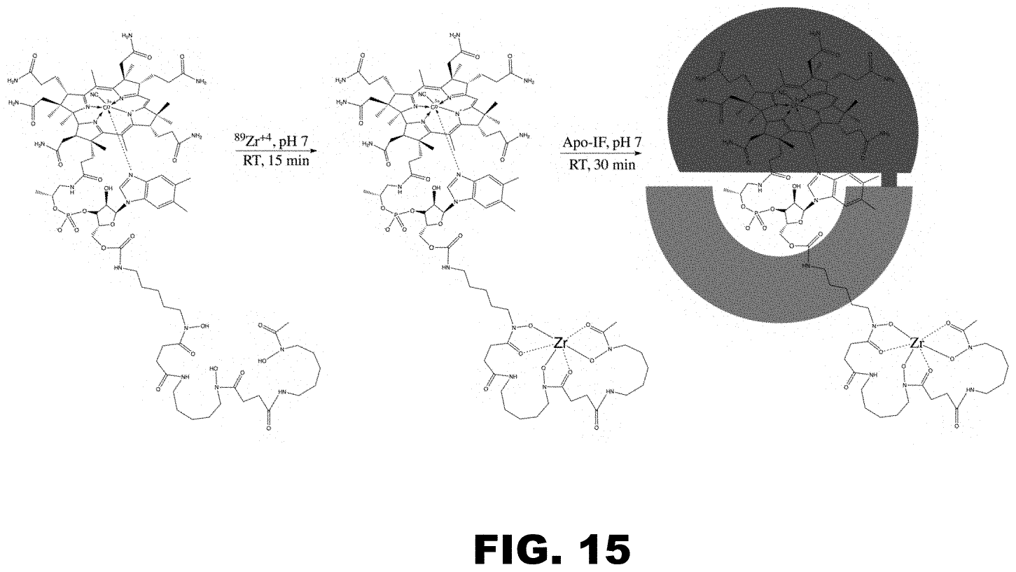

Synthesis of B12-DFO and B12-DFO-⁹Zr B12-desferrioxamine (B12-DFO) and B12-DFO-⁸⁹Zr (⁸⁹Zr-B12) were synthesized and characterized as previously reported.[30] Optimum conditions for radio labeling of B12-DFO were tested by titrating with ⁸⁹Zr and analyzing the incubated solution using iTLC. Approximately 1 mCi (37 MBq) of ⁸⁹Zr(C₂O₄)₂ (3D imaging, AZ) was diluted with 0.9% saline and the pH was adjusted to 7 by adding 1 M Na₂CO₃. A solution of B12-DFO (0.004 μmol, 10.8 μg) was added to the pH adjusted ⁸⁹Zr acetate solution and incubated for 15 min at room temperature (RT) (FIG. 15). The identity of the tracer was characterized via MALDI-MS analysis using B12-DFO labeled with ‘cold’ ⁹¹Zr⁴⁺ (FIG. 7), as control; Expected: 2030.2 [M⁺]; observed: 2005.2 [M-CN+H]⁺.

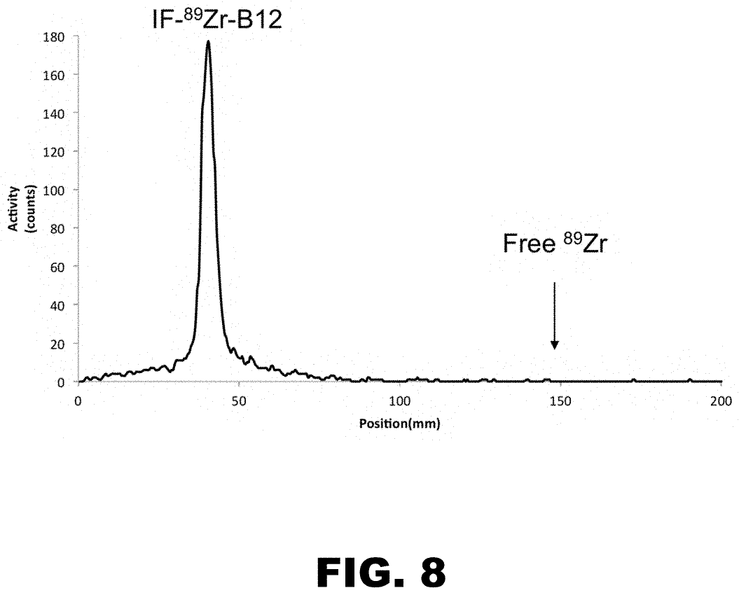

Binding ⁸⁹Zr-B12 to IF: A 1:0.8 ratio (apo-IF:⁸⁹Zr-B12-DFO) was used for binding. The radiolabeled compound was incubated with IF for 30 min at neutral pH at room temperature then purified through a 30 kDa size exclusion spin filter volume (GE Vivaspin) was adjusted with saline solution. Radio labeling efficiency of >97% was determined by iTLC (FIG. 8). Stability was confirmed over 24 hours in saline (FIG. 9).

Stability of IF-⁸⁹Zr-B12: Stability of IF-⁸⁹Zr-B12 was tested by incubating the tracer (200 μCi, 100 μl) in saline (0.9% NaCl) (Sigma) at 37° C. and fractions (50 μCi) were analyzed for free ⁸⁹Zr at 1, 4, and 24 h intervals using radio-HPLC (Agilent) and iTLC.

IF binding affinities: To confirm that ⁸⁹Zr-B12 will bind to IF, a radiometric chase assay, using ⁵⁷Co-B12 was completed (as previously reported)[15] with a cold tracer (⁹¹Zr-B12) and compared to free B12, as cyanocobalamin (CN-B12). Zr-B12 was made using B12-DFO and chelated to ZrCl₄ at pH 7.5.

Synthesis of B12-Cy5: B12-Cy5 was synthesized and characterized as previously reported.[3] Yield: 94%.

Flow cytometry measurements of cellular internalization: Cells were plated on a 6-well plate and allowed to adhere for at least 24 h until at least 80% confluency. Cells were washed 3× with HBSS and then incubated with 1 mL of IF-B12-Cy5, B12-Cy5 (200 nM) or HBSS without any conjugate unless otherwise indicated for 1 h and then washed in triplicate with HBSS. Cells were stripped mechanically and 1 mL of media was added and analysis performed. All cells were excited at 640 nm and detected at 660±20 nm.

GC-MS analyses of the glycosylation profile of recombinant human IF expressed in A. thaliana: Samples were analyzed by SGS M-Scan Inc. (West Chester, Pa., USA) by GC/MS. Key table generated in report is included as Table 1B.

PET imaging experiments: ⁸⁹Zr-B12 was intravenously administered (200-250 μCi/mouse, 0.8-1 nmol) in sterile saline in female nude mice on a B12-deplete (Envigo (Teklad) custom B12-free diet) or B12-replete diet (regular chow) for 3 weeks. A μPET scanner (Siemens Concord) was used for PET imaging and was initially completed at 1, 4, and 24 post-injection (p.i.) time points while the mice were anesthetized with 1-2% isoflurane (Baxter, Deerfield, Ill.) however, due to the similarity of the scans and background clearance, only 24 h p.i was used throughout the rest of the experiments. Images were reconstructed using filtered back projection algorithm. ASIPro VM™ software version 6.3.3.0 (Concord) was used to analyze the images to acquire volumes-of-interest expressed as % injected dose per gram of tissue (% ID/g).

Ex vivo distribution: The tissue distribution of ⁸⁹Zr-Cbl was studied by administering 10-25 μCi (0.04-0.1 nmol) of the tracer on the lateral tail vain of the rodent. Euthanasia via CO₂ asphyxiation was performed at 1, 4, and 24 h p.i.

Immunohistochemistry: Livers were excised from athymic nude female mice upon euthanizing via CO₂ asphyxiation, snap frozen in liquid nitrogen and embedded in optimal cutting temperature embedding medium (OCT, Skaura Finetek). The entire block was then frozen in liquid nitrogen and stored frozen at −80° C. Tumor blocks were moved to −20° C. 24 h prior to slicing. Livers were sliced into 5 μm sections (Leica CM 1850), mounted on positively charged slides (Fisher) and dried overnight at room temperature. Slides were fixed in precooled (−20° C.) acetone for 10 minutes and allowed to evaporate for 20 minutes. Endogenous activity was blocked with 0.075% H₂O₂ for 10 minutes. Subsequently, slides were incubated with 10% FBS in PBS for 1 h in a humidified chamber at room temperature. Liver tissues were incubated with antibodies for CD206 (Abcam anti-mannose receptor antibody ab64693) 1 h at room temperature in a humidified chamber. A histomouse Max broad spectrum DAB kit (Invitrogen) was used following manufacturers protocols for all following steps. Slides were scanned using a slide scanner (Leica SCN400) and visualized using Leica SCN400 image viewer software.

### References for the Example

1. Workinger, J. L. and Doyle, R. P. Vitamin B12: Advances and Insights, Chapter 14 vitamin b12 and drug development; CRC Press: Boca Raton, 2017
2. Równicki, M.; Wojciechowska, M.; Wierzba, A. J.; Czarnecki, J.; Bartosik, D.; Gryko, D.; Trylska, J. Vitamin B 12 as a Carrier of Peptide Nucleic Acid (PNA) into Bacterial Cells. Scientific Reports 2017, 7 (1), 7644.
3. Mietlicki-Baase, E. G.; Liberini, C. G.; Workinger, J. L.; Bonaccorso, R. L.; Borner, T.; Reiner, D. J.; Koch-Laskowski, K.; McGrath, L. E.; Lhamo, R.; Stein, L. M.; et al. A Vitamin B12 Conjugate of Exendin-4 Improves Glucose Tolerance without Associated Nausea or Hypophagia in Rodents. Diabetes, Obesity and Metabolism 2018, 20 (5), 1223-1234.
4. Prieto, L.; Rossier, J.; Derszniak, K.; Dybas, J.; Oetterli, R. M.; Kottelat, E.; Chlopicki, S.; Zelder, F.; Zobi, F. Modified Biovectors for the Tuneable Activation of Anti-Platelet Carbon Monoxide Release. Chem. Commun. 2017, 53 (51), 6840-6843.
5. Giedyk, M.; Jackowska, A.; Równicki, M.; Kolanowska, M.; Trylska, J.; Gryko, D. Vitamin B12 Transports Modified RNA into E. Coli and S. typhimurium Cells. Chem. Commun. 2018.
6. Sah, B.-R.; Schibli, R.; Waibel, R.; Boehmer, L. von; Blauenstein, P.; Nexo, E.; Johayem, A.; Fischer, E.; Müller, E.; Soyka, J. D.; et al. Tumor Imaging in Patients with Advanced Tumors Using a New 99mTc-Radiolabeled Vitamin B12 Derivative. J Nucl Med 2014, 55 (1), 43-49.
7. Ikotun, O. F.; Marquez, B. V.; Fazen, C. H.; Kahkoska, A. R.; Doyle, R. P.; Lapi, S. E. Investigation of a Vitamin B12 Conjugate as a PET Imaging Probe. ChemMedChem 2014, 9 (6),1244-1251.
8. Guo, W.; Deng, L.; Chen, Z.; Chen, Z.; Yu, J.; Liu, H.; Li, T.; Lin, T.; Chen, H.; Zhao, M.; et al. Vitamin-B12-Conjugated Sericin Micelles for Targeting CD320-Overexpressed Gastric Cancer and Reversing Drug Resistance. Nanomedicine 2018.
9. Zelder, F. Recent Trends in the Development of Vitamin B12 Derivatives for Medicinal Applications. Chem. Commun. 2015, 51 (74), 14004-14017.
10. Kräutler, B. Antivitamins B12-A Structure- and Reactivity-Based Concept. Chem. Eur. J. 2015, 21 (32), 11280-11287.
11. Proinsias, K. ó; Giedyk, M.; Gryko, D. Vitamin B12: Chemical Modifications. Chem. Soc. Rev. 2013, 42 (16), 6605-6619.
12. Fedosov, S. N.; Berglund, L.; Nexø, E.; Petersen, T. E. Sequence, S-S Bridges, and Spectra of Bovine Transcobalamin Expressed in Pichia Pastoris. J. Biol. Chem. 1999, 274 (37), 26015-26020.
13. Furger, E.; Fedosov, S. N.; Lildballe, D. L.; Waibel, R.; Schibli, R.; Nexo, E.; Fischer, E. Comparison of Recombinant Human Haptocorrin Expressed in Human Embryonic Kidney Cells and Native Haptocorrin. PLOS ONE 2012, 7 (5), e37421.
14. Mathews, F. S.; Gordon, M. M.; Chen, Z.; Rajashankar, K. R.; Ealick, S. E.; Alpers, D. H.; Sukumar, N. Crystal Structure of Human Intrinsic Factor: Cobalamin Complex at 2.6-Å Resolution. PNAS 2007, 104 (44), 17311-17316.
15. Stupperich, E.; Nexø, E. Effect of the Cobalt-N Coordination on the Cobamide Recognition by the Human Vitamin B12 Binding Proteins Intrinsic Factor, Transcobalamin and Haptocorrin. Eur. J. Biochem. 1991, 199 (2), 299-303.
16. Bonaccorso, R. L.; Chepurny, O. G.; Becker-Pauly, C.; Holz, G. G.; Doyle, R. P. Enhanced Peptide Stability Against Protease Digestion Induced by Intrinsic Factor Binding of a Vitamin B12 Conjugate of Exendin-4. Mol. Pharmaceutics 2015, 12 (9), 3502-3506.
17. Greibe, E.; Kornerup, L. S.; Juul, C. B.; Fedosov, S. N.; Heegaard, C. W.; Nexo, E. The Tissue Profile of Metabolically Active Coenzyme Forms of Vitamin B12 Differs in Vitamin B12-Depleted Rats Treated with Hydroxo-B12 or Cyano-B12. Br. J. Nutr. 2018, 120 (1), 49-56.
18. Hygum, K.; Lildballe, D. L.; Greibe, E. H.; Morkbak, A. L.; Poulsen, S. S.; Sorensen, B. S.; Petersen, T. E.; Nexo, E. Mouse Transcobalamin Has Features Resembling Both Human Transcobalamin and Haptocorrin. PLOS ONE 2011, 6 (5), e20638.
19. Green, R.; Allen, L. H.; Bjørke-Monsen, A.-L.; Brito, A.; Gubant, J.-L.; Miller, J. W.; Molloy, A. M.; Nexo, E.; Stabler, S.; Toh, B.-H.; et al. Vitamin B12 Deficiency. Nature Reviews Disease Primers 2017, 3, 17040.
20. Nielsen, M. J.; Rasmussen, M. R.; Andersen, C. B. F.; Nexø, E.; Moestrup, S. K. Vitamin B12 Transport from Food to the Body's Cells-a Sophisticated, Multistep Pathway. Nat Rev Gastroenterol Hepatol 2012, 9 (6), 345-354.
21. Andersen, C. B. F.; Madsen, M.; Storm, T.; Moestrup, S. K.; Andersen, G. R. Structural Basis for Receptor Recognition of Vitamin-B12-Intrinsic Factor Complexes. Nature 2010, 464 (7287),
22. Fyfe, J. C.; Madsen, M.; Højrup, P.; Christensen, E. I.; Tanner, S. M.; Chapelle, A. de la; He, Q.; Moestrup, S. K. The Functional Cobalamin (Vitamin B12)—Intrinsic Factor Receptor Is a Novel Complex of Cubilin and Amnionless. Blood 2004, 103 (5), 1573-1579.
23. Gherasim, C.; Lofgren, M.; Banerjee, R. Navigating the B12 Road: Assimilation, Delivery, and Disorders of Cobalamin. J. Biol. Chem. 2013, 288 (19), 13186-13193.
24. Lildballe, D. L.; Mutti, E.; Birn, H.; Nexo, E. Maximal Load of the Vitamin B12 Transport System: A Study on Mice Treated for Four Weeks with High-Dose Vitamin B12 or Cobinamide. PLOS ONE 2012, 7 (10), e46657.
25. Christensen, E. I.; Nielsen, R.; Birn, H. From Bowel to Kidneys: The Role of Cubilin in Physiology and Disease. Nephrol. Dial. Transplant. 2013, 28 (2), 274-281.
26. Tauris, J.; Christensen, E. I.; Nykjaer, A.; Jacobsen, C.; Petersen, C. M.; Ovesen, T. Cubilin and Megalin Co-Localize in the Neonatal Inner Ear. Audiol. Neurootol. 2009, 14 (4), 267-278.
27. Seetharam, B.; Christensen, E. I.; Moestrup, S. K.; Hammond, T. G.; Verroust, P. J. Identification of Rat Yolk Sac Target Protein of Teratogenic Antibodies, Gp280, as Intrinsic Factor-Cobalamin Receptor. J Clin Invest 1997, 99 (10), 2317-2322.
28. Schwartz, A. L. The Hepatic Asialoglycoprotein Receptor. CRC Crit. Rev. Biochem. 1984, 16 (3), 207-233.
29. Martinez-Pomares, L. The Mannose Receptor. Journal of Leukocyte Biology 2012, 92 (6),1177-1186.
30. Kuda-Wedagedara, A. N. W.; Workinger, J. L.; Nexo, E.; Doyle, R. P.; Viola-Villegas, N. 89Zr-Cobalamin PET Tracer: Synthesis, Cellular Uptake, and Use for Tumor Imaging. ACS Omega 2017, 2 (10), 6314-6320.
31. Fedosov, S. N.; Laursen, N. B.; Nexo, E.; Moestrup, S. K.; Petersen, T. E.; Jensen, E. O.; Berglund, L. Human intrinsic factor expressed in the plant Arabidopsis thaliana. European Journal of Biochemistry. 2003, 270 (16), 3362-3367.
32. Gazi, U.; Martinez-Pomares, L. Influence of the Mannose Receptor in Host Immune Responses. Immunobiology 2009, 214 (7), 554-561.
33. Allavena, P.; Sica, A.; Solinas, G.; Porta, C.; Mantovani, A. The Inflammatory Micro-Environment in Tumor Progression: The Role of Tumor-Associated Macrophages. Critical Reviews in Oncology/Hematology 2008, 66 (1), 1-9.
34. Garcia-Aguilar, T.; Espinosa-Cueto, P.; Magallanes-Puebla, A.; Mancilla, R. The Mannose Receptor Is Involved in the Phagocytosis of Mycobacteria-Induced Apoptotic Cells. J Immunol Res 2016, 2016.
35. Garcia-Aguilar, T.; Espinosa-Cueto, P.; Magallanes-Puebla, A.; Mancilla, R. The Mannose Receptor Is Involved in the Phagocytosis of Mycobacteria-Induced Apoptotic Cells https://www.hindawi.com/journals/jir/2016/3845247/abs/(accessed Jan. 23, 2019).
36. Paveley, R. A.; Aynsley, S. A.; Turner, J. D.; Bourke, C. D.; Jenkins, S. J.; Cook, P. C.; Martinez-Pomares, L.; Mountford, A. P. The Mannose Receptor (CD206) Is an Important Pattern Recognition Receptor (PRR) in the Detection of the Infective Stage of the Helminth Schistosoma Mansoni and Modulates IFNγ Production. International Journal for Parasitology 41 (13-14), 1335-1345.

### Example 2

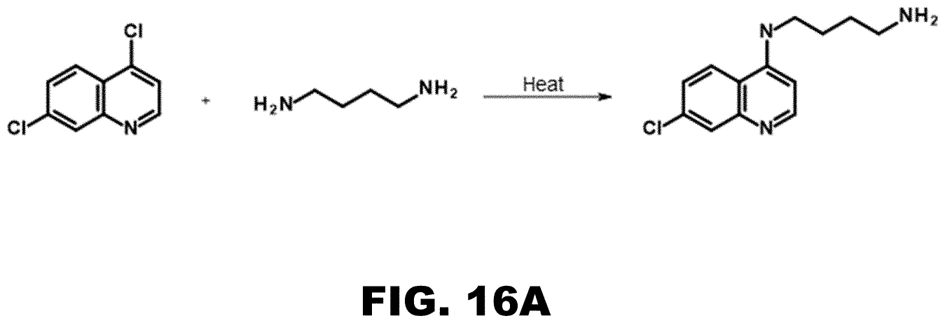

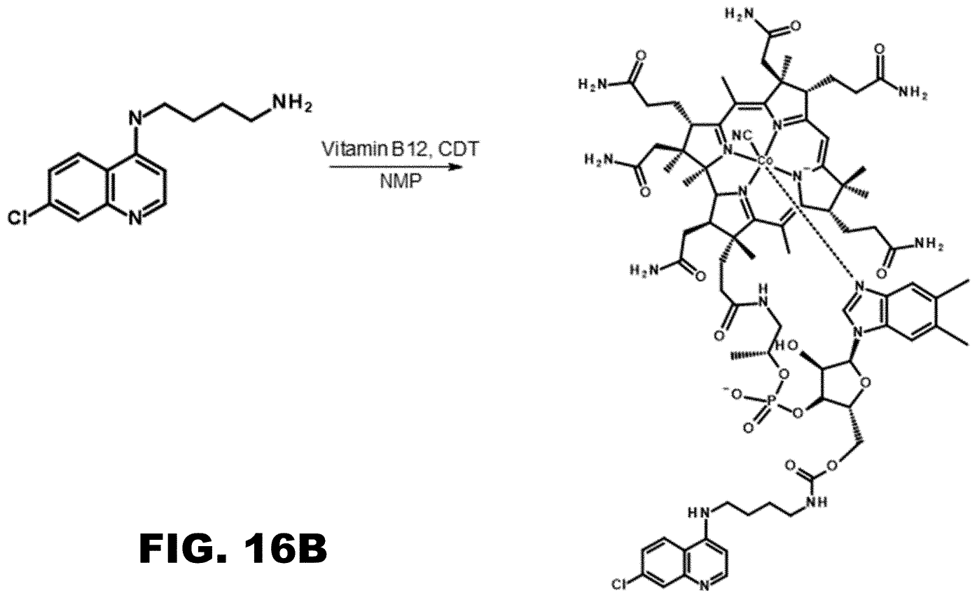

This example describes the preparation of a B12 conjugate comprising a chloroquine derivative. An overview of the synthetic process is depicted in FIG. 16.

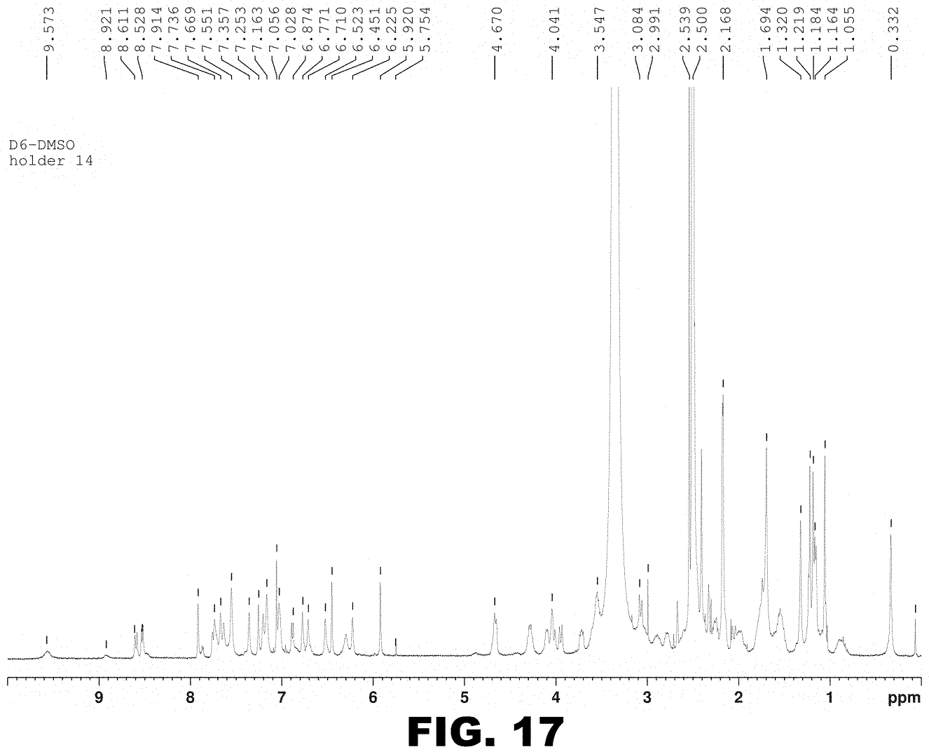

A mixture of 4,7-dichloroquinoline and diaminobutane was heated to 110° C. for 6 h under inert atmosphere and then cooled to room temperature (FIG. 16A). Aqueous NaOH (1N) was added and the mixture was extracted with CH₂Cl₂. The organic layers were washed with water, brine, dried over anhydrous Na₂SO₄ and evaporated under reduced pressure. The product was ready to use without further purification (FIG. 16B). To a 5 mL round bottom flask containing a stir bar, vitamin B12 was dissolved in dry NMP and allowed to stir at 40° C. under argon until starting material was fully dissolved. To the stirring solution of B12, 1,1′-carbonyl-di-(1,2,4-triazole) (CDT) was added and allowed to stir for one hour at which time the previously synthesized quinolineamine and triethylamine (TEA) were added and stirred until completion. Reaction completion was tracked via TLC. The reaction was then poured into ethyl acetate (AcOEt) (50 mL) and centrifuged (5 min, 4000 rpm, RT). The crude solid was redissolved in a minimal amount of methanol (MeOH) (≅5 mL), and precipitated with diethyl ether (Et2O), and centrifuged. The crude dry product was redissolved in 1 mL DI H₂O and purified utilizing RP-HPLC. Purity of product was determined via NMR (FIG. 17) and HPLC.

1. A pharmaceutical formulation for systemic administration, the pharmaceutical formulation comprising recombinantly produced intrinsic factor (IF), wherein the IF has a glycosylation pattern that enables binding to CD206, and is conjugated to a therapeutic, diagnostic, or imaging agent.

2. The pharmaceutical formulation of claim 1, wherein the IF is complexed to B12 or an analog thereof.

3. The pharmaceutical formulation of claim 1, wherein the IF is recombinantly produced in a plant.

4. (canceled)

5. The pharmaceutical formulation of claim 1, wherein the IF is glycosylated with α(1-3)-fucose, xylose, mannose and n-acetylglucosamine.

6. The pharmaceutical formulation of claim 5, wherein the IF is glycosylated with α(1-3)-fucose, xylose, mannose and n-acetylglucosamine the ratios of about 0.17: about 0.18: about 1.0: about 0.24, respectively.

7. (canceled)

8. The pharmaceutical formulation of claim 1, wherein the imaging agent is a radionuclide.

9.-19. (canceled)

20. A method of delivering a therapeutic, diagnostic, or imaging agent to a cell that expresses CD206 in a subject, the method comprising administering a pharmaceutical formulation of claim 1 to the subject.

21. The method of claim 20, wherein the cell is a liver cell or a macrophage.

22.-29. (canceled)

30. A method of delivering B12 to a cell that expresses CD206 in a subject, the method comprising administering a pharmaceutical formulation of claim 2 to the subject.

31. The method of claim 30, wherein the cell is a liver cell or a macrophage.

32. The method of claim 30, wherein the B12 is conjugated to an imaging agent and/or therapeutic agent.

33.-35. (canceled)

36. A pharmaceutical formulation, the pharmaceutical formulation comprising recombinantly produced intrinsic factor (IF) with a glycosylation pattern that enables binding to CD206; B12 or a B12 analog; and a therapeutic, diagnostic or imaging agent; wherein the B12 or B12 analog is conjugated to the therapeutic, diagnostic, or imaging agent.

37. The pharmaceutical formulation of claim 36, wherein the IF is complexed to B12 or an analog thereof.

38. The pharmaceutical formulation of claim 36, wherein the IF is recombinantly produced in a plant.

39. (canceled)

40. The pharmaceutical formulation of claim 36, wherein the IF is glycosylated with α(1-3)-fucose, xylose, mannose and n-acetylglucosamine.

41. (canceled)

42. The pharmaceutical formulation of claim 36, wherein the binding of IF to CD206 is not affected by endogenous B12 levels.

43. The pharmaceutical formulation of claim 36, wherein the imaging agent is a radionuclide.

44. (canceled)

45. The pharmaceutical formulation of claim 43, wherein the radionuclide is also a therapeutic agent.

46.-59. (canceled)

60. A method of delivering a therapeutic, diagnostic, or imaging agent to a cell that expresses CD206 in a subject, the method comprising administering a pharmaceutical formulation of claim 36 to the subject.

61. The method of claim 60, wherein the cell is a liver cell or a macrophage.

62.-80. (canceled)
